(19) United States
(12) Patent Application Publication
(10) Pub. No.: US 2023/0157317 A1
(43) Pub. Date: May 25, 2023

(54) AN ISOLATED BACILLUS ALTITUDINIS STRAIN AND ITS USE AS A PROBIOTIC

(71) Applicants: WATERFORD INSTITUTE OF TECHNOLOGY, Waterford (IE); TEAGASC - THE AGRICULTURE AND FOOD DEVELOPMENT AUTHORITY, Carlow (IE)

(72) Inventors: Gillian GARDINER, Cork (IE); Peadar LAWLOR, Cork (IE); Maria Luz PRIETO, Waterford (IE); Alan MARSH, Waterford (IE)

(21) Appl. No.: 17/910,656

(22) PCT Filed: Mar. 12, 2021
(86) PCT No.: PCT/EP2021/056425

§ 371 Date: Sep. 9, 2022

(30) Foreign Application Priority Data
Mar. 12, 2020 (EP) 20162860.9

(51) Int. Cl. A23K 10/18; C12N 1/20; A61K 35/742

(52) U.S. Cl. CPC A23K 10/18; A61K 35/742; C12N 1/205; C12R 2001/07

(57) ABSTRACT

An isolated Bacillus altitudinis strain as deposited with the National Collection of Industrial and Marine Bacteria under the Accession No. NCIMB 43558 on 27/01/2020.

### FIELD OF THE INVENTION

The invention relates to a seaweed-derived zootechnical additive. More specifically, the additive comprises spores, the supernatant, and/or the cell extract of Bacillus altitudinis, which can be provided to monogastric animals and mammals, and/or introduced to their environment, to improve growth in their offspring.

### BACKGROUND TO THE INVENTION

In commercial pig production, feed contributes up to 70% of total production costs. Such costs can be compounded by poor feed conversion caused by sub-optimal nutrition, infection, stress, and sub-optimal weight gain. This often necessitates costly dietary supplementation on a per animal basis, which is expensive and sometimes not efficacious. Furthermore, in 2006 the EU banned routine in-feed antibiotic use, and has also implemented a ban on the preventive use of antibiotics in groups of animals, and via medicated feed, and another ban on supplementation with pharmacological levels of zinc oxide, both of which will enter into force in 2022. This necessitates the development of alternative sustainable treatments and strategies to support development of a healthy piglet intestinal microbiota and optimal gut health. The availability of a clean-label feed additive that promotes robust and durable piglet growth would mean a significant increase in the output and value of saleable meat for commercial pig producers. The need for feed additives which can act as a replacement for pharmacological levels of zinc oxide is evidenced by the Zero Zinc Summit held in Denmark in June 2019, at which there were over 450 attendees.

Any of the EU-approved probiotics for sows, i.e. Bonvital (Lactosan GmbH & Co; Enterococcus faecium), Bioplus 2B (Christian Hansen; Bacillus subtilis and Bacillus licheniformis), Biosprint (Prosol; Saccharomyces cerevisiae), Calsporin (Asahi Calpis Wellness Co., Ltd; Bacillus subtilis), Clostat (Kemin; Bacillus subtilis) and Levucell SC (Lallemand Animal Nutrition; Saccharomyces cerevisiae), only demonstrate effects in the offspring during the early post-weaning period. These products were approved for use with sows for the full reproductive cycle, i.e. for the entire gestation period + ~28 days of lactation, and this lengthy administration period adds cost for the farmer. Three of the six products listed above are not spore formers and so would not be as resistant to drying and feed pelleting temperatures or as stable long-term in dried form as spores.

Feeding piglets with probiotics to achieve weight gain has been attempted before, with little success (Caisin and Harea, Animal Science and Biotechnologies, vol. 43(1), pp. 20-25 (2010); WO 2019/002476).

While the above has concentrated on pig production, the same problems are also prevalent in other monogastric animals and mammals, including non-ruminant/ruminant farm or farmed animals, non-ruminant/ruminant veterinary mammals and animals, and non-ruminant/ruminant mammals and animals kept in captivity, who are exposed to probiotics that are not effective in alleviating poor feed conversion and/or improving gut health.

It is an objective of the present invention to overcome at least one of the above-mentioned problems.

### SUMMARY OF THE INVENTION

The invention relates to a novel Bacillus altitudinis probiotic additive that addresses the challenge of maintaining the growth performance and herd health targets required for cost-effective pig production, without the use of in-feed antibiotics or pharmacological levels of zinc oxide. The proposed benefits include improved growth performance and increased resistance to disease e.g. post-weaning diarrhoea.

There is provided an isolated modified strain of a Bacillus altitudinis strain having a gyr B amplicon (fragment) sequence and/or a 16S rRNA amplicon (fragment) defined by SEQ ID NO: 1 and 2, respectively.

In one aspect, there is provided a cell extract or supernatant of an isolated modified strain of a Bacillus altitudinis strain having a gyr B amplicon (fragment) sequence and/or a 16S rRNA amplicon (fragment) defined by SEQ ID NO: 1 and 2, respectively.

In one aspect, there is provided a composition comprising the isolated modified Bacillus altitudinis strain having a gyr B amplicon (fragment) sequence and/or a 16S rRNA amplicon (fragment) defined by SEQ ID NO: 1 and 2, respectively, or the cell extract or supernatant of the isolated modified Bacillus altitudinis strain having a gyr B amplicon (fragment) sequence and/or a 16S rRNA amplicon (fragment) defined by SEQ ID NO: 1 and 2, respectively.

According to an aspect of the present invention, there is provided, as set out in the appended claims, an isolated Bacillus altitudinis strain as deposited with the National Collection of Industrial and Marine Bacteria under the Accession No. NCIMB 43558 on 27/01/2020 (hereafter “strain of the invention” or “deposited strain). This parent strain can be made antibiotic resistant by simple manipulation of its genome using techniques well-known to the skilled person. For example, the strain of the invention can be modified to be rifampicin resistant.

The invention also relates to a cell extract, a supernatant, spores or cell material derived from the isolated strain.

The invention also provides a composition comprising the isolated strain, or a cell extract, a supernatant, spores, or cell material derived from the isolated strain.

The composition may be a pharmaceutical composition and may include a suitable pharmaceutical excipient. The composition may be provided in a unit dose form suitable for oral and/or topical administration, i.e., a tablet, a capsule, a pellet, freeze-dried granules or powder, spray-dried granules or powder, nanoparticles, microparticles or in a liquid form.

The composition may be a food or beverage product, a food or beverage additive, a nutritional supplement, or an animal feed or drink additive (for example, in animal feed or pet food, or liquid refreshments (water, supplementary milk, and the like)). The products and additives are suitable for human ingestion and tailored to be suitable for non-human mammal and animal ingestion. The beverage in this instance can be drinking water, supplementary milk, or other liquid drinks provided to the mammal or animal.

The animal feed additive may be in powdered form, that may be added to animal feed or a feed pre-mix. Animal feed/feed pre-mix may comprise proteins, carbohydrates, fats, additional probiotics, prebiotics, vitamins, minerals, chemical preservatives, enzymes, immune modulators, milk replacers, amino acids, coccidiostats, acid-based products and/or medicines, such as antibiotics.

The animal feed additive comprising the composition of the claimed invention is also suitable for spraying onto feed pellets (before and/or after feed pelleting).

The animal feed additive may also comprise a suitable feed additive carrier, such as, anti-caking agents, anti-oxidation agents, bulking agents, and/or protectants.

Carbohydrate-containing components which may be used are for example forage, roughage, wheat meal, sunflower meal or soya meal, and mixtures thereof.

Protein-containing components are for example soya protein, pea protein, wheat gluten or corn gluten, and mixtures thereof.

Fat-containing components are, in particular, oils, of both animal and plant origin, like vegetable oils, for example soya bean oil, rapeseed oil, sunflower seed oil, flaxseed oil or palm oil, fish oil, and mixtures thereof.

Enzymes which may be used in feed compositions according to the invention and which may aid in the digestion of feed, are preferably selected from phytases (EC 3.1 .3.8 or 3.1.3.26), xylanases (EC 3.2.1.8), galactanases (EC 3.2.1 .89), galactosidases, in particular alpha-galactosidases (EC 3.2.1.22), proteases (EC 3.4), phospholipases, in particular phospholipases Al (EC 3.1 .1.32), A2 (EC 3.1.1.4), C (EC 3.1.4.3), and D (EC 3.1.4.4), lysophospholipases (EC 3.1 .1.5), amylases, in particular alpha- amylases (EC 3.2.1.1 ); lysozymes (EC 3.2.1 .17), glucanases, in particular beta-glucanases (EC 3.2.1.4 or EC 3.2.1.6), glucoamylases, cellulases, pectinases, or any mixture thereof.

Vitamins which may be used are for example vitamin A, vitamin D3, vitamin E, vitamin K, e.g., vitamin K3, vitamin B12, biotin, choline, vitamin B1, vitamin B2, vitamin B6, niacin, folic acid and pantothenate, e.g., Ca-D-pantothenate, or combinations thereof.

Amino acids which may be used according to the invention are for example lysine, alanine, threonine, methionine or tryptophan, or combinations thereof.

Further the isolated strain of the claimed invention preferably survives the high temperatures necessary for pelleting animal feed, in particular the strain preferably survives a temperature of 80° C., more preferably of 95° or 99° C., for at least 20 minutes.

In one aspect, the composition is added to drinking water, supplementary milk, or other liquid drinks.

The composition may comprise a prebiotic material, such as inulin, galacto-oligosaccharides, fructo-oligosaccharides, lactulose, mannose, maltose, mannan-oligosaccharides; malto-oligosaccharides, isomaltulose, palatinose, xylan, xylo-oligosaccharides, arabinoxylo-oligosaccharides, soy, soy polysaccharides, chito-oligosaccharides, cello-oligosaccharides, raffinose oligosaccharides, galactosyllactose, yeast cell wall extracts, seaweed extracts, laminarin, fucoidan, pectin, cellulose, resistant starch, plant polyphenols, polydextrose, β-glucans and the like.

The composition may comprise an additional probiotic bacterium, such as a Bacillus species, a Lactobacillus species, a Clostridium species, an Enterococcus species, E. coli, a Pediococcus species, and the like, and/or probiotic yeast, such as Saccharomyces cerevisiae or S. boulardii, and the like.

The strain in the composition may be viable or non-viable and may comprise a strain extract (i.e., bacterial cell lysate) or supernatant derived from the strain. The extract or supernatant may be in any physical form, for example liquid or dried.

The composition may comprise at least 10⁶ cfu per g of composition. In one aspect, the composition may comprise 1 × 10⁹ spores/ml or cfu/ml. Typically, the composition may comprise between 1 × 10⁶ to 1 × 10¹⁰ spores/ml or cfu/ml. For example, 1 × 10¹⁰ spores/ml or cfu/ml, 1 × 10⁷ spores/ml or cfu/ml, 1 × 10⁸ spores/ml or cfu/ml, 1 × 10⁹ spores/ml or cfu/ml, or 1 × 10¹⁰ spores/ml or cfu/ml.

The composition may be solid or liquid. The composition may comprise a carrier for oral delivery. The carrier may be in the form of tablet, capsule, powder, granules, microparticles or nanoparticles. The carrier may be configured for targeted release in the intestine (i.e., configured for gastric transit and ileal release).

The composition may be dried or lyophilised.

In one aspect, when the composition is suitable for topical or environmental administration, the composition comprises a carrier for topical or environmental administration selected from the group comprising powder, granules, microparticles, nanoparticles, a cream, a spray, spores, or liquid. The powder, granules, microparticles, nanoparticles and spray, as well as the spores themselves, can all be freeze-dried or spray-dried prior to application.

In one aspect, there is provided a composition described above for use in a method of increasing weight gain in offspring of a monogastric animal fed or exposed to the composition.

In one aspect, there is provided a composition described above for use in a method of improving the gastrointestinal health of a subject fed or exposed to the composition.

There is also provided a (pharmaceutical) composition for use in a method of increasing weight gain in offspring of a monogastric animal fed or exposed to the composition.

In one aspect, the (pharmaceutical) composition described above can be used in a method of improving the health of an individual. In one aspect, the health relates to gastrointestinal health.

There is also provided a method of increasing weight gain in an offspring of a lactating mammal, the method comprising providing the lactating mammal with the (pharmaceutical) composition described above during the entire gestation and during the lactating period.

In one aspect, the mammal is fed the composition during late gestation and during the lactating period.

In one aspect, the mammal is fed the composition during late gestation and for about the first 28 days of the lactating period.

In one aspect, the mammal is fed the composition for about the final 14 days of gestation and for about the first 28 days of the lactating period.

In one aspect, the composition is applied to the body of the lactating mammal for about the final 14 days of gestation and for about the first 28 days of the lactating period.

In one aspect, the composition is dispersed into the environment of the lactating mammal for about the final 14 days of gestation and for about the first 28 days of the lactating period.

In one aspect, the lactating mammal is selected from a human, primates, non-human primates, farm or farmed animals (such as pigs, horses, goats, sheep, cows (including bulls, bullocks, heifers etc.), donkey, reindeer, etc.), veterinary mammals and animals (such as dogs, cats, rabbits, hamsters, guinea pigs, mice, rats, ferrets, etc.), and mammals and animals kept in captivity (such as lions, tigers, elephants, zebras, giraffes, pandas, rhino, hippopotamus, etc.).

There is also provided a method of increasing weight gain in an offspring of a monogastric animal, the method comprising feeding the monogastric animal with the (pharmaceutical) composition described above during late gestation and for about the first 28 days following birth.

In one aspect, the composition is applied to the body of the monogastric animal for the entire gestation period and for about the first 28 days following birth or for about the final 14 days of gestation and for about the first 28 days following birth.

In one aspect, the composition is dispersed into the environment of the monogastric animal for the entire gestation period and for about the first 28 days following birth or for about the final 14 days of gestation and for about the first 28 days following birth.

In one aspect, the monogastric animal is selected from a human, primates, non-human primates, non-ruminant farm or farmed animals (such as pigs, horses, donkey, chicken, turkey, ducks, fish (salmon, trout, abalone), etc.), non-ruminant veterinary mammals and animals (such as dogs, cats, rabbits, hamsters, guinea pigs, mice, rats, ferrets, etc.), and non-ruminant mammals and animals kept in captivity (such as lions, tigers, elephants, zebras, pandas, rhino, hippopotamus, etc.).

There is also provided a method of increasing weight gain in an offspring of a bird, the method comprising injecting the (pharmaceutical) composition described above in ovo prior to hatching.

In one aspect, the (pharmaceutical) composition described above can be used in a method of improving the health of the offspring of the bird. In one aspect, the health relates to gastrointestinal health.

The bird is typically selected from an egg-laying hen, a wild turkey, a farmed turkey, a Guinea fowl, a goose, a duck, a canary, a budgerigar, a parrot, and the like.

The invention also provides a method of producing a supernatant from an isolated Bacillus altitudinis strain comprising a step of culturing the isolated strain and separating the supernatant from the strain.

The invention also provides a method of producing an extract from an isolated Bacillus altitudinis strain comprising a step of lysing the cell and separating the cell extract from lysed cell material.

The invention also provides a supernatant or bacterial material or extract (for example a cell lysate) formed according to the method of the invention.

Thus, a composition comprising spores from the B. altitudinis strain of the claimed invention can be used to improve the nutritional quality of colostrum (increased protein content), positively modulate the histology parameters of the intestine and modulate the intestinal and/or colostrum microbiome.

“Bacillus altitudinis strain” refers to the strain of bacteria deposited with the National Collection of Industrial and Marine Bacteria (Ferguson Building, Craibstone Estate, Bucksburn, Aberdeen AB219YA, UK) under the Accession No. NCIMB 43558 on 27/01/2020. The Deposit was made by the Waterford Institute of Technology, and having an address of Waterford Institute of Technology, Waterford, Ireland. The term is intended to include the strain in a viable or non-viable form, or mixtures of viable and non-viable bacteria. The strain may be provided in any format, for example as a liquid culture (or supernatant derived from a liquid culture), or cell material, spores or extract derived from the strain or a culture of the strain, or in a dried or freeze-dried format. The invention may also employ growth media in which the strain of the invention was grown, or cell lysates generated using the strain of the invention. The term also includes mutants, modified strains and variants of the deposited strain that are substantially identical, genetically and phenotypically, to the deposited strain and retain the activity of the deposited strain, such as introducing an antibiotic resistant gene (for example, rifampicin) to the strain. Thus, the term includes derivatives of the strain that have been genetically engineered to modify the genome of the strain, typically without fundamentally altering the functionality of the organism, for example engineered for heterologous expression of a nucleic acid, or engineered to overexpress an endogenous gene, or engineered to silence a gene, to produce a recombinant or transformed strain of the invention. Genetic modifications may be carried out using recombinant DNA techniques and reagents, including gene editing technologies such as CRISP-Cas9 techniques (see below). The term also includes variants of the strain having natural or spontaneous genetic alterations. The term is also intended to encompass variant strains obtained by serial passage of the isolated strain of the invention. The variant generally has a gyr B amplicon (fragment) sequence and/or a 16S rRNA amplicon (fragment) that is identical or substantially identical with the deposited strain, for example at least 99.1%, 99.2%, 99.3%, 99.4%, 99.5%, 99.6%, 99.7%, 99.8% or 99.9% identical to the deposited strain. Sequence homology can be determined using an online homology algorithm “BLAST”, publicly available at http://www.ncbi.nlm.nih.gov/BLAST/. The sequence of the gyr B amplicon (SEQ ID NO: 1) and the sequence of the 16S rRNA amplicon (SEQ ID NO: 2) for the Deposited Strain is provided in Annex 1 below.

### Dosage

It is preferable that the strain or composition is administered at least once per day over a treatment period of at least 6 weeks, and preferably for at least 7, 8, 9, 10, 11, 12, 13, 14, 15, 16, 17, 18- or 20-week periods. Compositions of the invention generally comprise between 10³ and 10¹² cfu or spores of the strain of the invention per g or per ml of the composition, respectively. In one embodiment, the composition comprises 10³ and 10¹² cfu or spores, or 10⁴ and 10¹² cfu or spores, or 10⁶ and 10¹⁰ cfu or spores of the strain of the invention per g or per ml of the composition, respectively. A daily dose generally comprises between 10³ and 10¹² cfu or spores per g or per ml of the strain, respectively. In one embodiment, the daily dose comprises10³ and 10¹² cfu or spores, or 10⁴ and 10¹² cfu, or 10⁶ and 10¹⁰ cfu or spores per g or per ml of the strain, respectively. In one aspect, the daily dose can be administered orally or by applying the composition to the environment in which the individual is residing, or to the skin or feathers of the adult animal or the offspring. For example, the composition could be applied to the udder of the lactating mammal or onto the offspring themselves once they are born. Another example is where the composition is applied to the environment within which the individual is residing.

After administering the strain described herein to gestating/lactating sows for a 6-week period (i.e., during the last 2 weeks of gestation and the ~28 days of lactation), residual growth benefits in offspring were observed during the finisher period and at slaughter, at which point improved carcass weight was found, which has financial benefits for pig producers.

The strain can also be used in other monogastric animal species, for example, chickens, as, like pigs, they are farmed intensively with the use of in-feed antibiotics.

### Definitions

In the specification, the term “healthy” should be understood to mean where the monogastric animal or mammal has no underlying medical condition, infection, inflammatory response, condition or otherwise occurring.

In the specification, the term “infection” or “infectious condition” should be understood to mean infections or diseases caused by microorganisms or microbes such as viruses, bacteria, fungi, protozoa, and helminths. Examples of infections include septicaemia, meningitis, eye infections, tuberculosis, upper respiratory tract infections, pneumonia, gastroenteritis, nail and skin infections and the like. Common bacteria that cause disease and infections include Staphylococcus, Streptococcus, Pseudomonas, Clostridium difficile, Salmonella, Campylobacter, Escherichia coli and Listeria monocytogenes.

In the specification, the term “inflammatory condition” should be understood to mean immune-related conditions resulting in allergic reactions, myopathies and abnormal inflammation and non-immune related conditions having causal origins in inflammatory processes. Examples include acne (in humans), asthma, autoimmune conditions, autoinflammatory conditions, celiac disease (in humans), chronic prostatitis, colitis, diverticulitis, glomerulonephritis, inflammatory bowel diseases, lupus, rheumatoid arthritis, vasculitis, cancer, heart disease, and the like.

In the specification, the term “individual” or “subject” should be understood to mean all monogastric animals and mammals and pre-ruminants (an animal that does not yet chew the cud), for example, a human, primates, non-human primates, farm or farmed animals (such as pigs, horses, goats, sheep, cows (including bulls, bullocks, heifers etc.), donkey, reindeer, chicken, turkey, ducks, fish (salmon, trout, abalone, etc.), veterinary mammals and animals (such as dogs, cats, rabbits, hamsters, guinea pigs, mice, rats, ferrets, etc.), and mammals and animals kept in captivity (such as lions, tigers, elephants, zebras, giraffes, pandas, rhino, hippopotamus, etc.), and other animals, mammals and higher mammals for which the use of the invention is practicable.

In the specification, the term “non-ruminant animal” should be understood to mean any mammal excluding cattle, all domesticated and wild bovines, goats, sheep, giraffes, deer, gazelles, and antelopes.

In this specification, the term “biological sample” should be understood to mean blood or blood derivatives (serum, plasma etc.), urine, saliva, skin/udder swabs, rectal swabs, nasopharyngeal swabs, vaginal swabs, faecal samples, gut digesta, gut tissue, colostrum, milk, or cerebrospinal fluid.

In the specification, the term “treatment” should be understood to mean prohibiting, preventing, restraining, and slowing, stopping, or reversing progression or severity of a disease or condition associated with inflammatory or non-inflammatory diseases, condition, or infections.

In the specification, the term “improving growth” should be understood to mean improving the weight gain in offspring of individuals where the individuals have been exposed to the strain of the present invention. By using the term “exposing”, we mean that the adult individuals have been fed the strain of the present invention or have had their body parts (such as udders, teats, feathers, brooding patch, skin, fur, etc.) sprayed with a composition comprising the strain, or the environment where the adults and offspring are residing is sprayed with the composition. While not bound by the theory, typically, there is transference of the strain from the adult to the offspring or from the environment to the offspring.

In the specification, the term “positively modulate” should be understood to mean having a positive effect on the beneficial microbiota/microbiome of the gut and having a positive effect on the health of the gut.

In the specification, the term “improve health” should be understood to mean elevate the health status of the individual to a better condition that it was prior to contacting the spores of the strain of the claimed invention or the composition comprising the spores of the strain of the claimed invention.

In the specification, “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the composition is administered. This relates to both feed additives and carriers for the composition. Such (pharmaceutical) carriers can be sterile liquids, such as water and oils, including those of petroleum, animal, vegetable or synthetic origin, such as peanut oil, soybean oil, mineral oil, sesame oil and the like. Water is a preferred carrier when the composition is administered when sprayed. Saline solutions and aqueous dextrose and glycerol solutions can also be employed as liquid carriers. Suitable excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like.

Suitable carriers for feed additives are inert formulation ingredients added to improve recovery, efficacy, or physical properties and/or to aid in packaging and administration. Such carriers may be added individually or in combination. These carriers may be selected from anti-caking agents, anti-oxidation agents, bulking agents, and/or protectants. Examples of useful carriers include polysaccharides (in particular starches, maltodextrins, methylcelluloses, gums, chitosan and/or inulins), protein sources (in particular skim-milk powder and/or sweet-whey powder), peptides, sugars (in particular lactose, trehalose, sucrose and/or dextrose), lipids (in particular lecithin, vegetable oils and/or mineral oils), salts (in particular sodium chloride, sodium carbonate, calcium carbonate, chalk, limestone, magnesium carbonate, sodium phosphate, calcium phosphate, magnesium phosphate and/or sodium citrate), and silicates (in particular clays, in particular beolite clay, amorphous silica, fumed/precipitated silicas, zeolites, Fuller’s earth, baylith, clintpolite, montmorillonite, diatomaceous earth, talc, bentonites, and/or silicate salts like aluminium, magnesium and/or calcium silicate).

The composition, if desired, can also contain minor amounts of wetting or emulsifying agents, or pH buffering agents. These compositions can take the form of solutions, suspensions, emulsion, tablets, pills, capsules, powders, sustained-release formulations and the like.

The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington’s Pharmaceutical Sciences” by E. W. Martin. The formulation should suit the mode of administration.

In the specification, the term “probiotic-supplemented” should be understood to mean supplemented with a probiotic comprising spores, or otherwise (cell extract supernatant, the strain itself), from the deposited strain of the claimed invention.

In the specification, the term “fed”, “feed” or “feeding” should be understood to mean composition is provided to the mammal or animal in a form that allows the composition (containing the spores of the strain of the claimed invention) or the spores themselves to be ingested. This is by typical oral administration vehicles such as in food or drink (for example, drinking water, supplementary milk or other liquid drinks).

### BRIEF DESCRIPTION OF THE DRAWINGS

The invention will be more clearly understood from the following description of an embodiment thereof, given by way of example only, with reference to the accompanying drawings, in which:-

FIG. 1 illustrates the trial layout for use of the deposited strain of the invention in pigs.

FIG. 2 illustrates the duodenal morphology of (A) a piglet euthanized on day 8 post-weaning (pw), born to a sow fed a basal diet supplemented with spores of the deposited strain of the claimed invention from day 100 of gestation until weaning (day 26 of lactation) compared to that of (B) a piglet born to a sow fed a basal (control) diet. The difference in villous height is indicated.

FIG. 3 shows the results from three sows fed with spores of the strain of the clamed invention over a 10-day period (starting on day 110 of gestation until day 3 of lactation), wherein samples (pen floor swabs, sow faeces, and udder swabs) were collected 7 days after the commencement of probiotic administration, i.e. on day 1 of lactation. Sampling was then performed for 3 consecutive days, i.e. day 1, day 2, and day 3 of lactation. The spores of the claimed invention were detected in (A) the farrowing room/pen environment, (B) on the udders of the sows, (C) and in the sow faeces. The unheated samples relate to the count of spores and the vegetative cells, while the heated samples relate to spore count only. FIG. 3A and FIG. 3B show that Bacillus spores were recovered from both pen floor and udder, while vegetative cells (germinated spores) were detected from pen samples but not from udder samples. This indicates that Bacillus spores can germinate in the farrowing room environment. FIG. 3C illustrates that there were higher spore and vegetative cell count than spore count at T0, T24 and T144 hr when sow faeces was incubated at a temperature simulating that in the farrowing room (i.e. vegetative cells present). This indicates that Bacillus spores can germinate in sow faeces. * P<0.05; ** P<0.01.

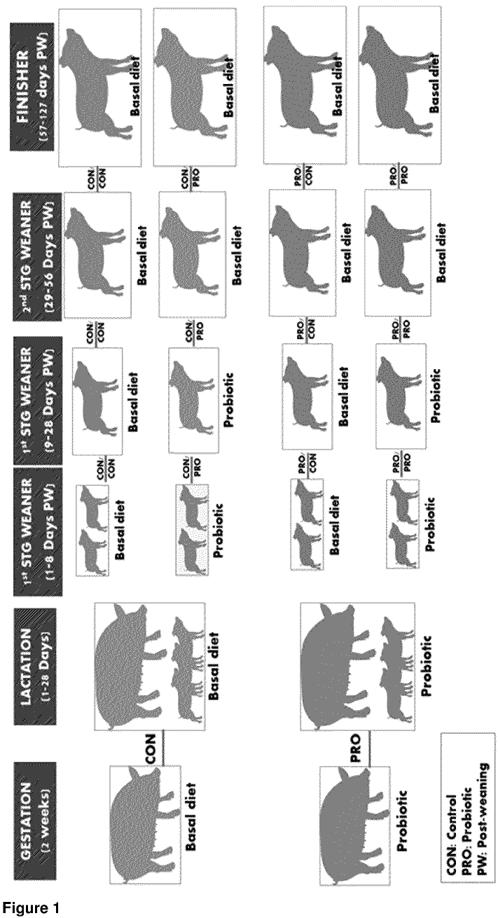

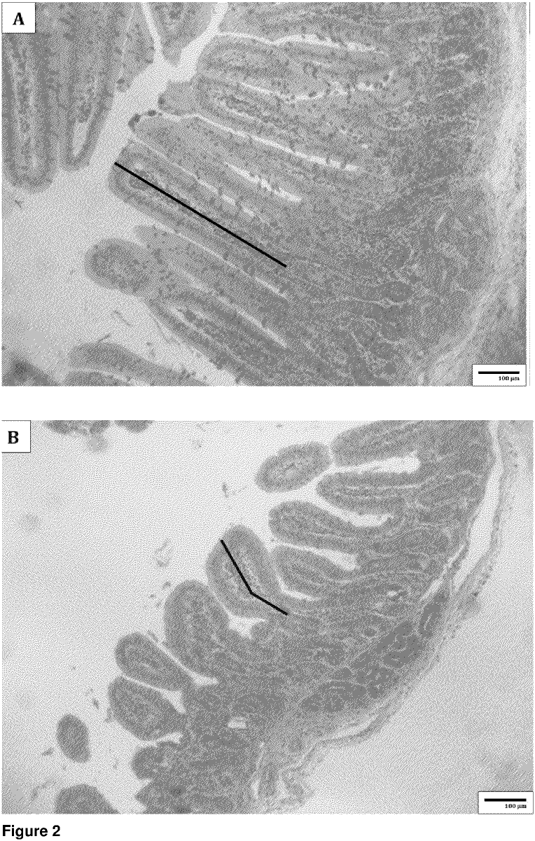

### DETAILED DESCRIPTION

### Materials and Methods

Ethical approval was granted by the Teagasc Animal Ethics Committee (approval no. TAEC148/2017) and the project was authorised by the Health Products Regulatory Authority (project authorisation no. AE19132/P066). The experiment was conducted in accordance with Irish legislation (SI no. 543/2012) and the EU Directive 2010/63/EU for animal experimentation. A total of 24 healthy sows were selected on day 100 of gestation, blocked on parity, body weight and back fat and randomly assigned to 2 experimental treatment groups (12 sows/group) as follows:
CON: control, basal gestation/lactation diet (n=12)
PRO: basal gestation/lactation diet supplemented with spores of the B. altitudinis strain of the claimed invention [~4 × 10⁹ spores/sow/day during gestation and ~1.2 × 10¹⁰ spores/sow/day during lactation (n=12)]

A total of 144 weaned piglets (-26 days of age) from the above 24 sows (n=12 sows/trt i.e., 6 piglets per sow) were blocked on litter origin, sex, and body weight, and randomly assigned to 4 experimental treatment groups following a 2 × 2 factorial arrangement where the main factors were probiotic inclusion in the sow diet (yes vs. no) and in 1^{st} stage weaner diets (yes vs. no). The four treatment groups were as follows:
CON/CON: piglets from CON sows fed basal weanling diet (n=18 pens)
CON/PRO: piglets from CON sows fed basal weanling diet supplemented with spores of the B. altitudinis strain of the claimed invention (~1 × 10⁹ spores/piglet/day) (n=18 pens)
PRO/CON: piglets from PRO sows fed basal weanling diet (n=18 pens)
PRO/PRO: piglets from PRO sows fed basal weanling diet supplemented with spores of the B. altitudinis strain of the claimed invention (~1 × 10⁹ spores/piglet/day) (n=18 pens)

The probiotic comprising the B. altitudinis strain of the present invention was fed at the optimum dose, as determined in a previous trial, and also taking into account doses used for commercial probiotics (Table 1). During the gestation period, sows were given ~4 × 10⁹ spores/day. For lactating sows, ~1.2 × 10¹⁰ spores/day were provided (Table 1). For 1^{st} stage weaning piglets (up to day 28 pw), ~1 × 10⁹ spores/day were given (Table 1). All pigs were followed to the end of the finisher period.

Feed and water were provided on an ad libitum basis during lactation and at 2.7 kg/day for the 10 days prior to parturition. Sows were fed with a standard gestation diet from day 100 of gestation to farrowing, followed by a standard lactation diet for 28 days (until weaning). The composition of the experimental diets is shown in Table 2. In the case of PRO sows, the diet was supplemented with spores of the B. altitudinis strain of the claimed invention from day 100 of gestation until weaning of piglets. Probiotic spores were administered daily. Spore suspensions were top dressed onto the feed as outlined below. The CON group received the same volume of sterile water top dressed onto the feed daily.

### Piglet Offspring

Piglets were penned as pairs for the first 7 days post-weaning. On day 8 pw, a total of 40 piglets (10 piglets per treatment) were sacrificed to determine intestinal probiotic counts and microbiota profile and to assess gut health. The paired piglets from the other pens were removed from the trial and the remaining piglets were individually housed until the end of the 2nd stage weaner period (day 55 pw). At day 56 pw, pigs were moved to finisher rooms where they were individually penned in fully slatted pens (1.81 m × 1.18 m) until the end of the trial (day 127 pw).

Feed and water were provided ad libitum. Starter/link diet were fed for the first 28 days after weaning. Weaner diet was provided from day 29 pw, followed by finisher diet from day 56 pw until slaughter. All feed was non-medicated. The composition of the experimental diets is shown in Table 2. During 1st stage weaning, pigs were fed with enough feed in the evening to ensure that they had sufficient feed overnight but that feeders were empty in the morning so that they immediately consumed the probiotic feed. This was practiced only during the 1st stage weaning period.

Probiotic spores were administered daily (after any sampling that took place) to treatment groups (CON/PRO and PRO/PRO) during the 1st stage weaner period only. A small amount of feed was put into the feeder and spore suspensions were top dressed onto the feed as outlined below. Any treatment groups not receiving probiotic (PRO/CON and CON/CON) received the same volume of sterile water top dressed onto the feed.

Growth performance of piglets was assessed by weighing pigs individually and monitoring individual feed intake in order to calculate average daily gain (ADG), average daily feed intake (ADFI), and feed conversion efficiency (FCE). Feed weigh-backs were recorded weekly and pigs were individually weighed as outlined below. Pigs were fasted for 12 hours prior to pre-slaughter weighing. Blood samples were taken at the same time points for haematological analyses to assess health.

### Hygiene Practices

### Farrowing Room

All farrowing rooms were thoroughly washed and disinfected a week before moving in the sows. Then one swab (blue sponge swab soaked in neutralising buffer; TS/15-A-95, Technical Service Consultants Ltd) was taken from each room for bacterial culture to check the absence of the probiotic strain in the farrowing rooms prior to entry of the sows.

During the experiment, every precaution was taken to prevent cross-contamination between treatments. Gloves were changed between pigs, and fresh disposable overalls worn by all personnel prior to commencing sampling. Particular care was taken to prevent the probiotic spores contaminating the control sows. Probiotic-fed sows were housed separately in two rooms. A foot bath containing 1% Virkon was placed outside each room to avoid contamination between rooms. Virkon solution was freshly prepared every Monday morning and Wednesday evening, or before, should it have lost its pink colour. Gloves and overalls were removed before leaving the probiotic farrowing room and put into a waste bag for immediate disposal.

To avoid contamination with probiotic spores, collection of samples, weighing of pigs and administration of distilled water/spores was done first with control sows followed by sows supplemented with spores. All equipment, e.g., weighing scales, snare used for blood sampling, etc., was disinfected thoroughly with Virkon after each treatment and sampling/weighing day to prevent cross contamination at subsequent sampling.

During blood sampling, puncture wounds were cleaned with alcohol prior to and after blood sampling.

### Sample Collection and Parameters Measured

### Sows

### Blood and Faecal Samples (N=24), Weighing (N=24) and Back Fat Scanning (N=24)

Blood and faecal samples were collected at the start of the trial (day 100 gestation), close to farrowing (day 114 and 115 of gestation, respectively), and at weaning of litters (-day 26 of lactation). A faecal sample was also taken at -day 13 of lactation. Body weight and back fat of all sows was recorded at the start of the trial (day 100 gestation), on the expected farrowing date (day 114 of gestation), and at weaning of litters (-day 26 of lactation).

### Colostrum and Milk (N=24)

Colostrum (15-20 ml or as much as possible) was collected from each sow within 12 hrs of farrowing. At day 14 of lactation, 40 ml of milk was collected from each sow.

### Piglets

### Blood Samples

At weaning, day 8 pw during sacrifice, day 28 pw and day 57 pw, blood samples were taken from 40 piglets (10 piglets/treatment).

### Faecal Samples

On day ~13 of lactation, rectal swabs were taken from a subset of suckling piglets (2 piglets/sow, n=48). Thereafter, faecal samples from 40 piglets (10 piglets/treatment) were collected from the rectum at weaning, at day 8 pw during sacrifice, at day 27 pw and at day 56 pw and naturally voided samples were collected from as many pigs as possible during the grow-finisher stage (day 118 pw).

### Digesta Samples

On day 8 pw during sacrifice, digesta samples were collected from the ileum and caecum from 40 piglets (10 piglets/treatment).

### Microbiome Analysis of Pig Faecal, Rectal and Digesta Samples by 16S rRNA Gene Sequencing

DNA was extracted from samples using the QIAamp DNA stool minikit (Qiagen, Crawley, United Kingdom) according to the manufacturer’s instructions, apart from adding a bead beating step after sample addition to the InhibitEX buffer and increasing the lysis temperature to 95° C. to increase the DNA yield. Microbial profiling at phylum, family, genus and ASV levels was performed using high throughput sequencing of the V3-V4 region of the 16S rRNA gene on an Illumina MiSeq platform according to standard Illumina protocols (McCormack et al., 2017, Exploring a possible link between the intestinal microbiota and feed efficiency in pigs. Applied & Environmental Microbiology 83(15): e00380-17).

### Microbiome Analysis of Colostrum Samples

DNA was extracted from colostrum samples using the DNeasy® PowerFood® Microbial Kit (Qiagen, Crawley, United Kingdom) according to the manufacturer’s instructions. Microbial profiling was performed as outlined above for faecal/rectal/digesta samples.

### Weighing

Body weight of all piglets was recorded at birth (day 0), during the lactation period (day 14) and at weaning (day 26) (N=144). At day 8 pw, body weight was recorded prior to sacrifice (N=40). Body weight was also recorded at days 14, 28, 56, 105 and 127 pw (N=72).

### Sacrifice (N=40)

On day 8 pw one pig per pen from each of the 10 pens selected per treatment (n ₌ 40) was sacrificed to collect blood, digesta and gut tissue samples.

### Carcass Data (N=72)

On day 127 pw, finisher pigs were slaughtered at a commercial abattoir. Carcass weight, kill out %, lean meat %, muscle depth and fat depth data were collected from the factory.

Diet Supplementation

TABLE 1

Spore doses for sows and piglets

| | | | | |
|---|---|---|---|---|
| Stage | Concentration (probiotic spores/ml) | Volume | Total Dose (probiotic spores/day) | Average of Commercial Doses (probiotic spores/day) |
| 1st Stage Weaners | 1 × 10⁹ | 1 ml | 1 × 10⁹ | 5.2 × 10⁸ |
| Gestating Sows | 1 × 10⁹ | 4 ml | 4 × 10⁹ | 2.4 × 10⁹ |
| Lactating Sows | 1 × 10⁹ | 12 ml | 1.2 × 10¹⁰ | 6.6 × 10⁹ |

TABLE 2

Composition of experimental diets (on an air-dry basis: kg/tonne unless otherwise stated)

| Item | Dry Sow | Lactating Sow | Starter/link | Weaner | Finisher |
|---|---|---|---|---|---|
| Barley | 753.02 | 269.81 | 62.86 | 257.58 | 384.67 |
| Wheat | 0 | 429.6 | 112 | 433.57 | 400 |
| Maize | 0 | 0 | 300 | 0 | 0 |
| Soybean meal | 89.62 | 196.65 | 255 | 187.92 | 183.01 |
| Soya hulls | 121.8 | 0 | 0 | 0 | 0 |
| Full fat soya | 0 | 0 | 70 | 50 | 0 |
| Lactoflo¹ | 0 | 0 | 100 | 0 | 0 |
| Skim milk powder | 0 | 0 | 25 | 0 | 0 |
| Soya oil | 11 | 66 | 40 | 40 | 9.69 |
| Lysine HCI | 2.19 | 4.47 | 5.14 | 5.02 | 3.75 |
| DL-Methionine | 0.58 | 1.35 | 2.62 | 1.85 | 0.93 |
| L-Threonine | 0.6 | 2.45 | 2.55 | 2.09 | 1.7 |
| L-Tryptophan | 0 | 0.71 | 0.97 | 0.27 | 0.15 |
| L-Valine | 0 | 2.34 | 0.26 | 0 | 0 |
| Vitamin and mineral mix | 1.5² | 1.5² | 3³ | 3³ | 1⁴ |
| Salt feed grade | 4 | 5 | 3 | 3 | 3 |
| Mono di-calcium phosphate | 6.49 | 8.5 | 9.5 | 4.6 | 1 |
| Limestone flour | 9.08 | 11.5 | 8 | 11 | 11 |
| Phytase⁵ | 0.1 | 0.1 | 0.1 | 0.1 | 0.1 |
| Analysed chemical composition | | | | | |
| Dry matter | 875 | 898 | 891 | 897 | 876 |
| Crude protein | 129 | 164 | 190 | 193 | 171 |
| Fat | 36.6 | 102.8 | 65.1 | 72.1 | 43.5 |
| Crude fibre | 72 | 26 | 30 | 27 | 31 |
| Neutral detergent fibre | 162 | 82 | 88 | 84 | 103 |
| Ash | 40 | 48 | 48 | 45 | 43 |
| Lysine | 8.2 | 11.5 | 15.0 | 13.0 | 11.0 |
| Methionine | 2.7 | 3.8 | 5.8 | 4.6 | 3.5 |
| Methionine and cysteine | 5.4 | 7.0 | 9.1 | 7.9 | 6.7 |
| Threonine | 5.5 | 8.3 | 10.1 | 8.6 | 7.7 |
| Tryptophan | 1.7 | 2.8 | 3.4 | 2.6 | 2.3 |

Calculated chemical compostition⁶

| | | | | | |
|---|---|---|---|---|---|
| Standardised ileal digestible lysine | 6.60 | 10.67 | 14.00 | 11.49 | 9.97 |
| Calcium | 7.20 | 8.32 | 8.00 | 7.25 | 6.59 |
| Digestible phosphorus | 3.45 | 3.88 | 4.44 | 3.32 | 2.55 |
| Digestible energy (MJ/kg) | 13.2 | 15.2 | 15.0 | 14.5 | 13.8 |
| Net energy (MJ/kg) | 8.9 | 10.9 | 10.74 | 10.55 | 9.80 |

¹ Lactoflo 70 contains 70% lactose, 11.5% protein, 0.5% oil, 7.5% ash and 0.5% fibre (Volac, Cambridge, UK).
² Premix provided per kg of complete diet: Cu, 15 mg; Fe, 70 mg; Mn, 62 mg; Zn, 80 mg; I, 0.6 mg; Se, 0.2 mg; vitamin A, 1000 IU; vitamin D₃, 1000 IU; vitamin E, 100 IU; vitamin K, 2 mg; vitamin B₁₂, 15 µg; riboflavin, 5 mg; nicotinic acid, 12 mg; pantothenic acid, 10 mg; choline chloride, 500 mg; biotin, 200 mg; folic acid, 5 g; vitamin B₁, 2 mg; vitamin B₆, 3 mg.
³ Premix provided per kg of complete diet: Cu, 155 mg; Fe, 90 mg; Mn, 47 mg; Zn, 120 mg; l, 0.6 mg; Se, 0.3 mg; vitamin A, 6000 IU; vitamin D₃, 1000 IU; vitamin E, 100 IU; vitamin K, 4 mg; vitamin B₁₂, 15 µg; riboflavin, 2 mg; nicotinic acid, 12 mg; pantothenic acid, 10 mg; choline chloride, 250 mg; vitamin B₁, 2 mg; vitamin B₆, 3 mg; Endox, 60 g.
⁴ Premix provided per kg of complete diet: Cu, 15 mg; Fe, 24 mg; Mn, 31 mg; Zn, 80 mg; l, 0.3 mg; Se, 0.2 mg; vitamin A, 2000 IU; vitamin D₃, 500 IU; vitamin E, 40 IU; vitamin K, 4 mg; vitamin B₁₂, 15 µg; riboflavin, 2 mg; nicotinic acid, 12 mg; pantothenic acid, 10 mg; vitamin B₁, 2 mg; vitamin B₆, 3 mg.
⁵ The diet contained 500 phytase units (FYT) per kg feed from RONOZYME HiPhos (DSM, Belfast, UK).
⁶ Calculated from tabulated ingredient values: Sauvant D, Perez J-M & Tran G (editors) (2004) Tables of composition and nutritional value of feed materials. The Netherlands: Wageningen Academic Publishers.

### Compositional Analysis of Sow Colostrum and Milk Samples

Colostrum and milk samples were defrosted at room temperature. When fully thawed, samples were mixed by inverting several times to disrupt settled solids and mixed well. The volume of each sample was recorded prior to decanting into 50 ml tubes on ice. Sterile water was added to bring up the volume to 40 ml. Tubes were mixed thoroughly and kept on ice. Each sample was analysed in duplicate for total solids, lactose, fat, protein, true protein, and casein B content by near-infrared absorption using a Bentley Dairyspec FT (Bentley Instruments, Inc., Chaska, MN, USA). Data were recorded as % (g/100 g), taking the dilution factor into account.

### Intestinal Histology

Duodenal, jejunal, and ileal tissue samples were rinsed in phosphate buffered saline immediately post-harvest and placed in No-Tox, an alcohol/aldehyde fixative (Scientific Device Lab, Des Plaines, IL) on a shaker for 48 hr. Samples were then dehydrated through a graded alcohol series, cleared with xylene, and embedded in paraffin wax. Tissue samples were sliced into 5 micrometre sections using a microtome (Leica RM2135, Wetzlar, Germany), mounted on microscope slides and stained with hematoxylin and eosin for determination of gross morphological parameters of intestinal structure (villus height and width and crypt depth and width). For each pig, 10 villi and 10 crypts were measured on five fields of view, where villi were attached to the lumen, and the means were utilised for statistical analysis. The goblet cell number was determined by periodic acid-Schiff staining. Positively stained periodic acid-Schiff cells were enumerated on 10 villi/sample, and the means were utilised for statistical analysis.

### Haematological Analysis of Blood Samples

Haematological analysis was performed on whole blood using an Abbot Cell-Dyn 3700 analyser (GMI-Inc, Minnesota, USA). The following parameters were measured; white blood cell (WBC) number, lymphocyte number and percentage, monocyte number and percentage, granulocyte number and percentage, eosinophil number and percentage, basophil number and percentage, red blood cell (RBC) number, haemoglobin, mean corpuscular volume, mean corpuscular haemoglobin, platelets and packed cell volume.

### Statistical Analysis

Power calculations were performed to determine the minimum number of observations required to detect effect sizes, using a statistical power of 80%, an α level at 5% and standard deviation of variables of interest from 7 previously published studies. The power calculation indicated that 12 sows per treatment were required to see a difference of 2.5 mm in back fat depth, 10 piglets were required to see a 2 log10 CFU/g difference in selected microbial counts between treatments and that 18 piglets were required to see a 1.5 Iog10 CFU/g difference in microbial counts between treatments.

The experiment was a 2 × 2 factorial arrangement, with the factors being maternal treatment (control or probiotic supplementation) and post-weaning treatment (control or probiotic supplementation). All data were analysed using the MIXED procedure in SAS® 9.4 (SAS Institute, Inc., Cary, NC, US), unless otherwise stated. The model included maternal treatment and post-weaning treatment as fixed effects and their interaction. Where required, data were analysed as a repeated measure with sampling day as the repeated variable and the appropriate covariance structure, as indicated by the model fit statistics, was fitted to the data. Simple main effects were obtained using the ‘slice’ option in SAS.

The sow/litter was the experimental unit for sow performance, sow haematology, sow probiotic count data and colostrum and milk composition data. The individual pig was the experimental unit for analysis of pre- and post-weaning pig growth performance, carcass characteristics, haematology, small intestinal morphology and probiotic count data. The normality of scaled residuals was investigated using the Shapiro-Wilk and Kolmogorov-Smirnov tests within the UNIVARIATE procedure of SAS. Differences in least square means were investigated using the t-test after Tukey adjustment for multiple comparisons. Degrees of freedom were estimated using Satterthwaite adjustment.

For sow performance, litter size, and pre-weaning mortality data, block was included as a random effect. The initial value (day 100 of gestation) was included as a covariate in the analysis when significant in the model. Pre-weaning performance was analysed as repeated measures, including sex (male, female) as a fixed effect and block as a random effect. Birth weight was included as a covariate when significant in the model. Post-weaning performance was analysed as repeated measures, including sex (male, female) as fixed effect and weaning weight as a covariate, when significant in the model. For carcass characteristics, sex (male, female) was included as a fixed effect and body weight at weaning was included as a covariate when significant in the model. Counts of the strain of the claimed invention were analysed as repeated measurements. For the faecal counts of the strain of the claimed invention in the sows, block was included as a random effect. For the faecal counts of the strain of the claimed invention in the post-weaned piglets, the count at weaning was included as a covariate in the analysis, when significant. Haematological parameters were analysed including the initial value (day 100 of gestation for sows or day 0 pw for the offspring) as a covariate in the analysis when significant in the model. In addition, block was included as a random effect for the haematological values of sows. The haematological parameters that were not normally distributed were further analysed to find the best fitting distribution using the GLIMMIX procedure in SAS, using a gamma distribution. For these variables, the ilink function was used to back-transform the data to the original scale. The small intestinal morphology data were analysed using sex (male, female) as a fixed effect.

The results are presented in the text and tables as the least square means together with the pooled standard errors of the mean. Differences between treatments were considered significant for P<_0.05, while 0.05<P<_0.10 was considered as a tendency.

### Evaluate the Effects of Spores of the Probiotic Strain B. Altitudinis of the Claimed Invention Administered to Piglets During 1st and/or 2nd Stage Weaner Periods

The objective of this study was to evaluate the effects of spores of the deposited strain B. altitudinis of the claimed invention administered during the 1st and/or 2nd stage weaner periods on growth performance, health indicators and gut microbiota composition of pigs in comparison to an antibiotic (AB) zinc oxide (ZnO) combination. A total of 80 piglets (40 male and 40 female) were selected one day prior to weaning, blocked by sex, weight and litter origin, and randomly assigned to one of five experimental treatments as follows: 1) Negative control (no spores of the deposited strain B. altitudinis of the claimed invention) fed from day 0-56 pw (Neg/Neg); 2) Control diet with spores of the deposited strain B. altitudinis of the claimed invention fed from day 29-56 pw (Neg/Pro); 3) Control diet with spores of the deposited strain B. altitudinis of the claimed invention fed from day 0-28 pw (Pro/Neg); 4) Control diet with spores of the deposited strain B. altitudinis of the claimed invention fed from day 0-56 pw (Pro/Pro) and 5) Control diet containing ZnO at 2500 mg Zn/kg feed and AB (200 mg Apramycin /kg feed) from day 0-28 pw. The spores of the deposited strain B. altitudinis of the claimed invention were fed at ~1 × 10⁹ spores/day from day 0 to 28 pw and ~2 × 10⁹ spores/day from day 29 to 56 pw. All pigs were followed until the end of the finisher period (day 106 pw) to monitor residual effects of treatments.

### Results and Discussion

The concept is a novel seaweed-derived zootechnical additive. It comprises spores, supernatant, cell material of the deposited strain of the invention, which when fed to sows during late gestation and lactation, resulted in improved growth in the offspring.

A feeding trial compared the effects of this Bacillus feed additive on progeny growth when administered to sows over a ~6-week period (last 2 weeks of gestation and the ~28 days of lactation) against a control treatment (feed without the additive). Sows and offspring from both groups were continuously monitored and biological samples were collected from offspring at regular intervals from birth to slaughter. It was found that piglets born to sows fed the additive demonstrated faecal shedding of the strain while suckling, thereby demonstrating transfer of the microbial additive from sow to offspring. These piglets also exhibited the following benefits:
Numerically reduced pre-weaning mortality (10.1 vs 15.6% in offspring from control sows; P ₌ 0.183) and because of this there was a numerical increase in the number of piglets weaned per litter (11.8 vs 12.6; P=0.29) in response to probiotic supplementation of sows.
Better feed conversion ratio (FCR) for the first 14 days pw (1.28 vs 1.45 in offspring from control sows; P < 0.001). Commercially, this is important as feed contributes up to 70% of total production costs. Based on the fact that pigs eat on average ~3.0 kg feed in the first 14 days pw, the 0.17 reduction in FCR means that pigs from probiotic-supplemented sows will eat ~510 g less feed during this period which will save ~46 cents/pig given the price of feed is ~€0.895/kg. This represents a considerable cost-saving for farmers. However, and much more important, is the fact that an improvement in FCR at this time can be considered a proxy for improved intestinal health at weaning which can be correlated with increased lifetime growth.
Numerical improvements in body weight were found up to day 56 pw in offspring from probiotic-supplemented sows (+0.2, +0.7 and +0.6 kg on day 14, 28 and 56 pw, respectively; P>0.05).
After this, offspring were followed through the finisher stage to slaughter to monitor residual growth effects. Significant improvements in finisher pig live-weight were observed, with offspring from probiotic-supplemented sows 3.5 kg heavier at day 105 pw (P ₌ 0.013) and 3.5 kg heavier at day 127 pw (P ₌ 0.012).
Due to an increase in kill out yield for offspring from probiotic-fed sows (P=0.002) the carcass weight of offspring from probiotic-supplemented sows was 3.5 kg heavier than that of offspring from control sows (P ₌ 0.045). This translates to a financial benefit of €5.39 more per pig (at a 5-year average pig price of €1.54/kg carcass weight). This equates to €144.50 more income per sow per year, based on the fact that a sow produces an average of 26.8 piglets/year (Teagasc, 2019). Based on a typical 500-sow unit, this equates to a benefit of €72,495 a year from probiotic supplementation of sows.
Benefit to cost analyses were also performed using costs for some competitor probiotics as we do not know what the production costs will be for our probiotic at this stage. Two probiotics approved for use in sows were selected; Calsporin, which is relatively cheap at €1.50/tonne of finished feed, and Levucell, which is more expensive, at €5/tonne of finished feed. These costs and average sow feed intake data were firstly used to estimate the cost of feeding probiotic during the 6-week feeding period that was used (i.e. 2 weeks gestation + 4 weeks lactation) OR the entire reproductive cycle, i.e. entire gestation and lactation period (which is what these probiotics are recommended/approved for) as follows:
   Based on the cost of Calsporin, it would cost 82c per sow per year to feed probiotic for the 6-week period and €2.10 for the full reproductive cycle.
   Based on the cost for Levucell, it would cost €2.73 per sow per year for a 6-week administration period and €7 per sow for the full reproductive cycle.

Bearing in mind that feeding the probiotic of the claimed invention to sows would generate €144.50 more per sow per annum as outlined above, the benefit to cost was then estimated as follows:
Based on the cost of Calsporin and probiotic administration for 6 weeks, benefit to cost = 177:1, whereas if fed for the whole reproductive cycle, it would be 69:1. This essentially means that if adopting the 6-week administration period, for every €1 spent, a farmer could expect to get €177 back and for the full reproductive cycle, this would be €69.
Based on the cost of Levucell, this would be 53:1 for the 6-week period and 21:1 for the full reproductive cycle i.e., for every €1 spent, a farmer would expect to get back €53 or €21, respectively.

There was no impact of probiotic supplementation to offspring on body weight, ADG, or feed intake, post-weaning or during the finisher period and there was no impact on carcass weight or quality. However, there was a tendency for a post-weaning treatment effect on FCR from day 57 to day 105 pw and during the entire post-weaning period (day 0-127 pw), with probiotic-supplemented pigs having a better FCR than CON pigs [2.21 vs 2.13 (P=0.06) and 2.07 vs 2.04 (P=0.07), respectively].

In piglets born to sows fed probiotic, faecal probiotic shedding was detected while the piglets were suckling (see Table 3 below), even though probiotic had not been administered to the piglets themselves, demonstrating transfer from sow to offspring, which could offer a potentially cost-effective means of probiotic delivery to piglets in early life. This group of piglets shed probiotic at day 14 and 27 of age (i.e. while still suckling the sow). In fact, the faecal probiotic count in these piglets at day 27 of age was log₁₀ 4.79 cfu/g while in piglets supplemented with probiotic for the entire 1st stage weaner period it was log₁₀ 5.9 cfu/g at day 28 pw. This indicates that the probiotic had transferred successfully from the sow to offspring and as such this could prove to be a cost-effective means of probiotic delivery to piglets early in life.

TABLE 3

Counts of the deposited Bacillus altitudinis strain of the claimed invention in faeces (log₁₀ cfu/g) of sows and their piglets during gestation and lactation, where sows were fed a basal diet (Control) or a basal diet supplemented with spores of the deposited strain B. altitudinis (Probiotic) from day (D) 100 of gestation until weaning (D26 of lactation)^{1,2}

| | Treatment | | | | P-value | |
|---|---|---|---|---|---|---|
| Time point | Control | Probiotic | SEM³ | Treatment | Day | Treatment × Day |
| Sows | | | | | | |
| N | 12 | 12 | | | | |
| D100 of gestation | 3.00 | 3.00 | - | - | | - |
| D115 of gestation | 3.04 | 5.87 | 0.047 | <0.001 | | |
| D13 of lactation | 3.00 | 6.3 | 0.047 | <0.001 | | |
| Weaning (D26 of lactation) | 3.00 | 6.17 | 0.047 | <0.001 | | |
| Overall | | | 0.034 | <0.001 | <0.001 | <0.001 |

| | | | | | | |
|---|---|---|---|---|---|---|
| Piglets during lactation | | | | | | |
| N | 20 | 20 | | | | |
| D13⁴ | 3.00 | 3.47 | 0.075 | <0.001 | | |
| D26⁵ (weaning) | 3.00 | 4.79 | 0.080 | <0.001 | | |
| Overall | | | 0.055 | <0.001 | <0.001 | <0.001 |

¹ Least square means with their standard errors.
² The limit of detection was log₁₀ 3.00 cfu/g faeces. Values below the limit of detection were recorded as log₁₀ 3.00 cfu/g.
³ SEM: standard error of the mean.
⁴ Counts are from rectal swabs and are presented as Log₁₀ cfu/swab.
⁵ Rectal swabs were taken from three pigs in the probiotic treatment group due to insufficient faecal sample. Probiotic was detected in these animals, but the counts were excluded from the statistical analysis.

Growth benefits were seen in offspring from sows fed probiotic during late gestation and lactation, which translate to carcass weight increases and hence have considerable commercial significance, as outlined above (see also Table 4 below). A unique aspect of this is that offspring were monitored all the way through to slaughter (see Table 5 below) and so it is possible to demonstrate residual benefits to pig carcass weight, while any of the EU-approved probiotics for sows only demonstrate effects during the early post-weaning period.

TABLE 4

Post-weaning growth performance of piglets born to sows fed a basal diet (Control) or a basal diet supplemented with spores of the deposited strain B. altitudinis (Probiotic) from day (D) 100 of gestation until weaning (D26 of lactation)¹

| | | Maternal treatment | | | |
|---|---|---|---|---|---|
| | Day (pw²) | Control | Probiotic | SEM³ | P-value |
| N | | 36 | 36 | | |
| Mortality⁴ | | 1 | 0 | | |
| Off trial⁵ | | 4 | 2 | | |
| Body weight (kg) | 0 | 8.41 | 8.24 | 0.251 | 0.622 |
| | 14 | 11.38 | 11.55 | 0.922 | 0.893 |
| | 28 | 18.07 | 18.73 | 0.929 | 0.616 |
| | 56 | 42.43 | 42.98 | 0.943 | 0.680 |
| | 105 | 91.73 | 95.22 | 0.981 | 0.013 |
| | 127 | 121.01 | 124.51 | 0.973 | 0.012 |
| | Overall | | | 0.426 | 0.006 |
| Average daily gain (g) | 0-14 | 214.47 | 220.96 | 17.621 | 0.795 |
| | 15-28 | 483.53 | 514.14 | 17.749 | 0.224 |
| | 29-56 | 863.85 | 868.0 | 18.021 | 0.871 |
| | 57-105 | 1024.35 | 1065.93 | 18.755 | 0.118 |
| | 106-127 | 1334.01 | 1369.97 | 18.594 | 0.173 |
| | Overall | | | 8.135 | 0.040 |
| | 0-127 | 889.96 | 922.27 | 10.905 | 0.042 |
| Average daily feed intake (g) | 0-14 | 292.61 | 277.28 | 29.695 | 0.715 |
| | 15-28 | 620.83 | 642.44 | 29.913 | 0.610 |
| | 29-56 | 1272.92 | 1273.73 | 30.366 | 0.985 |
| | 57-105 | 2231.43 | 2293.68 | 31.586 | 0.165 |
| | 106-127 | 3251.94 | 3322.64 | 31.320 | 0.112 |
| | Overall | | | 13.696 | 0.150 |
| | 0-127 | 1834.79 | 1883.60 | 23.747 | 0.154 |
| Feed conversion ratio | 0-14 | 1.45 | 1.28 | 0.030 | <0.001 |
| | 15-28 | 1.30 | 1.26 | 0.030 | 0.350 |
| | 29-56 | 1.47 | 1.47 | 0.030 | 0.946 |
| | 57-105 | 2.18 | 2.16 | 0.032 | 0.682 |
| | 106-127 | 2.45 | 2.46 | 0.031 | 0.908 |
| | Overall | | | 0.014 | 0.019 |
| | 0-127 | 2.06 | 2.05 | 0.014 | 0.412 |

¹ Least square means with their standard errors.
² pw: post-weaning.
³ SEM: standard error of the mean.
⁴ Mortality: Due to polyserositis and septicaemia (Streptococcus suis infection).
⁵ Off trial: Pigs were removed from the trial due to lameness (Probiotic, N=1), pneumonia (Control, N=2), bloody diarrhoea (Control, N=1; Probiotic, N=1) and abdominal hernia (Control, N=1).

TABLE 5

Carcass characteristics of finisher pigs slaughtered at day 127 post-weaning, born to sows fed a basal diet (Control) or a basal diet supplemented with spores of the deposited strain B. altitudinis (Probiotic) from day (D) 100 of gestation until weaning (D26 of lactation)¹
Maternal treatment

| | Control | Probiotic | SEM² | P-value |
|---|---|---|---|---|
| N | 31 | 34 | | |
| Carcass weight (kg) | 90.89 | 94.43 | 1.218 | 0.045 |
| Kill out (%) | 75.04 | 75.91 | 0.187 | 0.002 |
| Lean meat (%) | 54.20 | 54.29 | 0.332 | 0.857 |
| Muscle (mm) | 48.22 | 50.75 | 1.138 | 0.122 |
| Fat (mm) | 15.54 | 15.94 | 0.426 | 0.512 |

¹ Least square means with their standard errors.
² SEM: standard error of the mean.

The probiotic strain decreased the lactose content of the sow colostrum (P<0.01) and increased protein, true protein, and casein content (P<0.05) but had no impact on milk composition (Table 6). The increased protein content of the colostrum may help to explain how the probiotic beneficially impacts offspring growth when administered to sows, as it would have increased nutritional value.

TABLE 6

Colostrum and milk (day 14 post-partum) analysis of sows fed a basal diet (Control) or the basal diet supplemented with probiotic spores from the deposited strain of the claimed invention (Probiotic).

| | Control | Probiotic¹ | SEM | P-value | Reference values² | Reference values³ |
|---|---|---|---|---|---|---|
| N | 12 | 12 | | | | |
| Colostrum (%) | | | | | | |
| Total solids | 21.97 | 24.01 | 0.581 | 0.021 | 17.2 - 30.2 | 24.8 |
| Lactose | 2.06 | 1.52 | 0.128 | 0.008 | 2.4 - 4.3 | 3.4 |
| Fat | 3.94 | 4.14 | 0.430 | 0.748 | 4.8 - 11.6 | 5.9 |
| Protein | 14.25 | 16.56 | 0.759 | 0.043 | 3.3-19.7 | 15.1 |
| True Protein | 13.83 | 16.18 | 0.791 | 0.047 | | |
| Casein B | 11.98 | 14.08 | 0.717 | 0.050 | | |
| Milk (%) | | | | | | |
| Total solids | 18.91 | 18.56 | 0.500 | 0.627 | 18.2 - 22.0 | 18.7 |
| Lactose | 5.23 | 5.22 | 0.130 | 0.930 | 5.1 - 6.3 | 5.3 |
| Fat | 7.42 | 6.74 | 0.524 | 0.368 | 5.3 - 10.8 | 7.6 |
| Protein | 4.78 | 4.89 | 0.116 | 0.493 | 4.7 - 7.1 | 5.5 |
| True Protein | 4.25 | 4.41 | 0.115 | 0.337 | | |
| Casein B | 3.34 | 3.47 | 0.111 | 0.445 | | |

¹ Probiotic: sows fed a basal diet supplemented with probiotic spores from D100 of gestation and the entire lactation period.
² Adapted from Hurley et al., 2015. Composition of sow colostrum and milk. In The Gestating and Lactating Sow; Farmer, C. (ed.), Wageningen Academic Publishers.
³ Darragh, A. J. & Moughan, P. J. 1998. The composition of colostrum and milk. In The Lactating Sow; Verstegen, M. W. A., Moughan, P. J. & Schrama, J.W. (eds.), Wageningen Academic Publishers.

Villous height and area were also increased in the duodenum of the offspring from probiotic-supplemented vs control sows (P ₌ 0.002 and 0.001, respectively) at day 8 pw and a tendency for increased villous height was observed in the ileum (P ₌ 0.07) (see Table 7 and FIG. 2). These findings indicate increased absorptive capacity in the small intestine early post-weaning, which may help to explain the improved FCR of the offspring of probiotic-treated sows during the first 14 days post-weaning and the subsequent growth benefits.

TABLE 7

Small intestinal morphology of pialets euthanized on D8 post-weaning, born to sows fed a basal diet (Control) or a basal diet supplemented with spores of the deposited strain of the claimed invention (Probiotic) from day (D) 100 of gestation until weaning (D26 of lactation)¹

| | Maternal Treatment | | | |
|---|---|---|---|---|
| | Control | Probiotic | SEM² | P-value |
| N | 20 | 20 | | |
| Duodenum | | | | |
| Goblet cells (number) | 13.8 | 14.9 | 1.14 | 0.52 |
| Villous height (µm) | 351.8 | 392.7 | 8.61 | <0.01 |
| Crypt depth(µm) | 177.0 | 190.5 | 4.43 | 0.04 |
| VH:CD³ ratio | 2.1 | 2.1 | 0.07 | 0.62 |
| Villous area (µm²) | 40888 | 48962 | 1814.2 | <0.01 |
| Crypt area (µm²) | 6739 | 7485 | 269.3 | 0.06 |
| Jejunum | | | | |
| Goblet cells (number) | 8.7 | 10.2 | 0.85 | 0.20 |
| Villous height (µm) | 346.3 | 362.8 | 8.07 | 0.16 |
| Crypt depth(µm) | 175.9 | 189.1 | 4.44 | 0.04 |
| VH:CD ratio | 2 | 2 | 0.06 | 0.37 |
| Villous area (µm²) | 38947 | 42105 | 1961.2 | 0.26 |
| Crypt area (µm²) | 6731 | 8075 | 343.7 | <0.01 |
| Ileum | | | | |
| Goblet cells (number) | 13.7 | 15.9 | 1.27 | 0.22 |
| Villous height (µm) | 325.7 | 345.8 | 7.39 | 0.06 |
| Crypt depth(µm) | 183.1 | 187.0 | 3.98 | 0.50 |
| VH:CD ratio | 1.8 | 1.9 | 0.05 | 0.41 |
| Villous area (µm²) | 37552 | 41677 | 1724.3 | 0.10 |
| Crypt area (µm²) | 7211 | 7659 | 290.6 | 0.28 |

¹ Least square means with their standard errors.
² SEM, standard error of the mean.
³ VH:CD: Villous height:crypt depth.

What is also interesting to note in a study to evaluate the effects of spores of the deposited B. altitudinis strain administered during 1st and/or 2nd stage weaning on growth performance, health indicators and gut microbiota composition of pigs in comparison to an antibiotic (AB) zinc oxide (ZnO) combination is that the deposited strain was shed in the faeces of all pigs fed the strain. Furthermore, the strain was still detected in 9/10 pigs sampled 7 days post-administration of the strain, albeit at very low levels. Body weight at day 106 pw was higher in the AB+ZnO treatment group compared to the Pro/Pro group (P<0.05). The AB+ZnO treatment group also had higher ADG (P<0.05) than the Pro/Neg group between day 0 and 14 pw, and day 15 and 28 pw. Overall, the ADG and ADFI were higher in the AB+ZnO treatment group than in the Pro/Neg group (P<0.05). Between day 0 and 14 pw, Gain:Feed (G:F) of the AB+ZnO treatment group was higher than that of all other treatments (P<0.01). Also during this period, G:F of the Pro/Pro treatment group was lower than that of the AB+ZnO treatment group but higher than that of Pro/Neg. None of the treatments impacted faecal scores post-weaning or carcass characteristics at slaughter. Some small effects of the probiotic treatments were observed for some of the haematological parameters measured; however, all values were within the normal ranges reported for pigs of the same age. Hence, it was assumed that these results were not of biological significance. 16S rRNA gene sequence-based profiling of the gut microbiome indicated that dietary inclusion of AB+ZnO had the greatest impact on gut bacterial populations, with large shifts occurring for some bacterial taxa, although the composition appeared to return to baseline within a few weeks of ceasing treatment. Inclusion of the probiotic spores appears to have had a more subtle, but beneficial, impact on the intestinal microbiota composition. The only probiotic-mediated effect on bacterial taxa found at >1 % relative abundance within the faecal microbiota was in pigs administered probiotic during the first 28 days post-weaning (Pro/Neg) and only then at day 35 pw. In these pigs, relative abundance of the Spirochaetes phylum and the Spirochaetaceae family was lower than in the Neg/Neg group (3.11% vs 9.19% and 3.15% vs 9.54%, respectively), likely resulting from a lower abundance of the Treponema genus (3.13% vs 9.88%). Ruminococcaceae-UCG-005 was also less abundant (1.36% vs 2.84%). The reduction in Treponema, a genus with known pathogenic species, may be beneficial as it is negatively correlated with body weight and ADG in pigs. On the other hand, this finding, along with the reduction in Ruminococcaceae, could be considered detrimental, as these taxa are associated with better feed efficiency in pigs; however, the Pro/Neg treatment did not have poorer feed efficiency at this time point. Three butyrate-producing genera were more abundant in the Pro/Neg group than the Neg/Neg group; Agathobacter (5.11% vs 1.71%), Faecalibacterium (1.63& vs 0.6%) and Roseburia (1.39% vs 0.51%). These increases can be considered beneficial, as butyrate has anti-inflammatory properties, prevents mucosal atrophy, is an energy source for the colonocytes, and a lack of butyrate-producing bacteria may cause dysbiosis in the gut. Overall, the results of this study show that inclusion of spores of the deposited strain of the claimed invention in the diet during 1st and/or 2nd stage weaner period did not affect piglet growth during the post-weaning or finisher periods (day 0-106 pw), although inclusion during the 1st stage weaner period favourably modulated the intestinal microbiota.

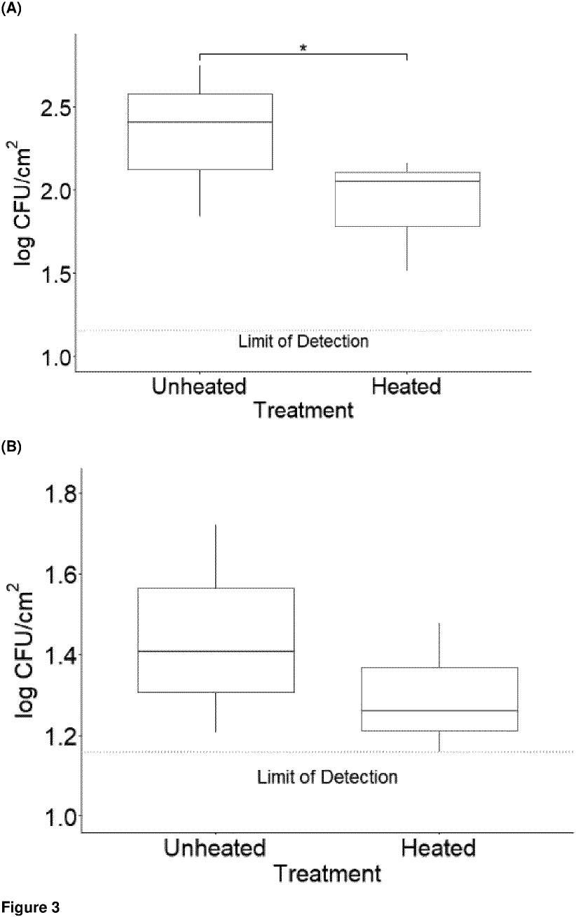

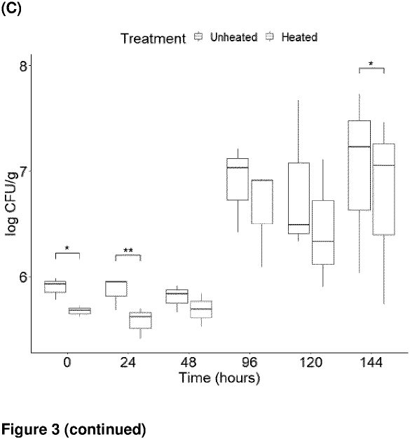

Looking at FIG. 3, the inventors have shown that the spores of the strain of the claimed invention were detected in swabs taken from the farrowing room floor where the sows were housed (FIG. 3A), and also in swabs from the sows’ udders (FIG. 3B). Furthermore, the vegetative cells were detected on the pen floor indicating that the spores had germinated in the farrowing room environment. When faeces from sows fed the spores was incubated at 22° C. to simulate the temperature of the farrowing room, FIG. 3C shows that the spores were also able to germinate and also that the spores persisted for up to 5 days.

These findings indicate that the spores persist in sow faeces under conditions simulating those in the farrowing room and so could also be expected to persist in the farrowing room environment thereby acting as a source of probiotic for suckling piglets. In addition, the fact that the spores germinate into vegetative cells could have benefits for offspring health. This is because the vegetative cells may be picked up by the suckling piglets and, because their gastric pH is higher than in weaned pigs, the vegetative cells could survive intestinal transit. The vegetative cells may be more beneficial in the gut as they are the metabolically active form.

The probiotic also modulated the microbiome of the sow’s colostrum and the intestinal microbiome of the offspring in a favourable manner.

### Haematological Parameters of Sows During Gestation and Lactation

The results for haematological parameters where there were significant treatment differences are reported in Table 8. There was a tendency for a treatment × day interaction for mean corpuscular haemoglobin concentration (P=0.09), which decreased on day 114 of gestation in CON sows, increasing again at weaning (day 26 of lactation). The only treatment difference found for blood cell counts was for basophils. Overall, PRO sows had a higher basophil count than CON sows (P<0.01). This was also found on day 114 of gestation (P=0.04) and a tendency for this effect was found on the day of weaning (day 26; P=0.07). Similar results were found for the overall percentage of basophils, where PRO sows had higher levels than CON sows (P=0.001). This was also found on day 114 of gestation (P=0.05) and on the day of weaning (day 26; P<0.01). Regarding the other parameters measured, treatment differences were also observed for mean corpuscular volume and mean corpuscular haemoglobin. Overall, CON sows had higher mean corpuscular volume than PRO sows (P<0.001) and this was also found on day 114 of gestation (P=0.001) and on the day of weaning (day 26; P<0.01). Overall, CON sows had greater mean corpuscular haemoglobin levels than PRO sows (P =0.001) and this was also found on day 114 of gestation (P=0.01) and at weaning (day 26; P=0.001). In addition, the mean corpuscular haemoglobin concentration was higher for PRO sows than for CON sows on day 114 of gestation (P=0.04).

TABLE 8

Effect of supplementing sow diets with spores from the deposited strain of the claimed invention from 100 of gestation to weaning (day 26 of lactation) on haematological parameters of sows¹.

| | | Treatment | | | P⁻value | | |
|---|---|---|---|---|---|---|---|
| Blood parameters | Day | CON² | PR0³ | SEM | Treatment | Day | Treatment x Day |
| N | | 12 | 12 | | | | |
| Basophils (×10³ cells/µL) | G100 | 0.10 | 0.11 | 0.013 | 0.54 | | |
| | G114 | 0.11 | 0.17 | 0.024 | 0.04 | | |
| | W26 | 0.17 | 0.22 | 0.022 | 0.07 | | |
| | Mean | 0.14 | 0.20 | 0.018 | <0.01 | <0.01 | 0.72 |
| Basophils (%)⁴ | G100 | 1.11 | 1.36 | 0.127 | 0.19 | | |
| | G114 | 1.24 | 1.81 | 0.207 | 0.05 | | |
| | W26 | 1.58 | 2.32 | 0.188 | <0.01 | | |
| | Mean | 1.41 | 2.06 | 0.155 | 0.001 | 0.02 | 0.61 |
| Mean corpuscular volume (fL) | G100 | 63.52 | 62.77 | 0.601 | 0.25 | | |
| | G114 | 66.18 | 63.88 | 0.474 | 0.001 | | |
| | W26 | 65.01 | 63.23 | 0.431 | <0.01 | | |
| | Mean | 65.60 | 63.55 | 0.357 | <0.001 | 0.03 | 0.51 |
| Mean corpuscular haemoglobin (pg/cell) | G100 | 19.90 | 19.57 | 0.197 | 0.13 | | |
| | G114 | 20.47 | 19.93 | 0.154 | 0.01 | | |
| | W26 | 20.20 | 19.54 | 0.139 | 0.001 | | |
| | Mean | 20.34 | 19.74 | 0.113 | 0.001 | 0.02 | 0.65 |
| Mean corpuscular haemoglobin concentration (g/dL) | G100 | 31.33 | 31.14 | 0.220 | 0.56 | | |
| | G114 | 30.89 | 31.23 | 0.122 | 0.04 | | |
| | W26 | 31.02 | 31.00 | 0.111 | 0.91 | | |
| | Mean | 30.96 | 31.12 | 0.093 | 0.14 | 0.62 | 0.09 |

G100: Day 100 of gestation; G114: day 114 of gestation; W26: weaning (day 26 of lactation).
¹ Least square means and pooled standard errors of the mean (SEM).
² CON: non-probiotic supplemented sows;
³ PRO: probiotic-supplemented sows.
⁴ Percentages are based on the differential count of white blood cells.

### Haematological Parameters of Pigs Post-Weaning

The effects of Bacillus altitudinis (the strain of the claimed invention) supplementation to sow and piglet diets on the haematological parameters of pigs post-weaning are shown in Table 9. No maternal treatment × post-weaning treatment × day interactions were found for any of the parameters measured, except for mean corpuscular volume (P=0.08) and mean corpuscular haemoglobin (P=0.09) which tended to decrease with increasing age in the pigs.

Pigs on the post-weaning PRO treatment had higher WBC counts on day 57 pw than CON pigs (14.62 vs 11.68 ± 0.962 × 10³ cells/µL; P=0.04). There was a tendency for a maternal treatment x post-weaning treatment interaction for the total lymphocyte count on day 57 pw (P=0.10). An effect of post-weaning treatment was found for the total number of lymphocytes and lymphocyte percentage at day 57 pw, where PRO pigs had a higher lymphocyte count and percentage than CON pigs [10.97 vs 7.29 ± 1.145 × 10³ cells/µL (P=0.03) and 68.03 vs 59.33 ± 2.954 % (P=0.04), respectively]. Similarly, the overall lymphocyte count and lymphocyte percentage tended to be higher in PRO compared to CON pigs [10.61 vs 8.42 ± 0.822 × 10³ cells/µL (P=0.06) and 68.95 vs 61.11 ± 2.135 % (P=0.01), respectively].

A maternal treatment x post-weaning treatment interaction was found on day 8 pw for monocyte count (P<0.01), with counts lower in the CON/CON group than in the PRO/CON group. Likewise, a tendency for a maternal treatment × post-weaning treatment interaction was also found for the percentage of monocytes on day 8 pw (P=0.09), with piglets from the CON/CON group having a lower percentage than their PRO/CON counterparts. This led to offspring from PRO sows having a higher monocyte percentage than pigs born to CON sows at day 8 pw (6.65 vs 4.76 ± 0.667 %; P=0.05). In addition, pigs on the post-weaning probiotic treatment had a lower percentage of monocytes than CON pigs on day 57 pw (7.95 vs 10.65 ± 0.873 %; P=0.03) and overall (6.36 vs 8.28 ± 0.631 %; P=0.04).

A maternal treatment x post-weaning treatment interaction was observed at weaning for the neutrophil count (P=0.05), where pigs from the CON/PRO group had a higher count than PRO/PRO pigs. A tendency for a post-weaning treatment effect was observed overall for the neutrophil percentage, where probiotic-supplemented pigs had a lower percentage of neutrophils than CON pigs (21.90 vs 26.90 ± 1.877 %; P=0.07).

There was a maternal treatment × post-weaning treatment interaction for both the eosinophil count (P=0.01) and percentage (P=0.001) on day 57 pw, with pigs from the PRO/CON group having a higher eosinophil count and percentage than pigs from the CON/PRO and PRO/PRO groups. A post-weaning treatment effect was also observed, with probiotic-supplemented pigs having lower eosinophil counts than CON pigs on day 8 pw (0.11 vs 0.16 ± 0.017 × 10³ cells/µL; P=0.03), day 57 pw (0.15 vs 0.22 ± 0.019 × 10³ cells/µL; P<0.01), and overall (0.15 vs 0.19 ± 0.014 × 10³ cells/µL; P=0.050). Similarly, probiotic-supplemented pigs had a lower eosinophil percentage than CON pigs on day 57 pw (0.95 vs 1.89 ± 0.140 %; P<0.001) and overall (0.97 vs 1.47 ± 0.102 %; P=0.001).

A maternal treatment x post-weaning treatment interaction was found for basophil count (P=0.001) and percentage (P=0.02) on day 8 pw, with CON/CON pigs having a lower basophil count and percentage than pigs from CON/PRO and PRO/CON groups. In addition, pigs born to CON sows had a lower basophil count than those born to PRO sows at weaning (0.07 vs 0.12 ± 0.012 × 10³ cells/µL; P=0.05) and day 8 pw (0.04 vs 0.06 ± 0.006 × 10³ cells/µL; P=0.02). This led to offspring from CON sows having a lower basophil percentage than those from PRO sows at weaning (0.58 vs 1.16 ± 0.108 %; P=0.01) and day 8 pw (0.37 vs 0.55 ± 0.058 %; P=0.03). An effect of post-weaning treatment was also observed for basophil percentage overall, where probiotic-supplemented pigs had a lower percentage than CON pigs (1.56 vs 2.07 ± 0.179 %; P=0.05).

At weaning, tendencies for a maternal treatment effect were observed for RBC count (7.82 vs 6.98 ± 0.318 × 10⁶ cells/µL; P=0.07), haemoglobin (15.08 vs 13.64 ± 0.594 g/dL; P=0.10) and haematocrit (0.50 vs 0.45 ± 0.018 L/L; P=0.05), with offspring from CON sows having higher levels than those from PRO sows. A tendency for a maternal treatment × post-weaning treatment interaction was observed for mean corpuscular haemoglobin at weaning (P=0.10), day 57 pw (P=0.08) and overall (P=0.07), and for mean corpuscular haemoglobin concentration overall (P=0.06). On day 8 pw, PRO-supplemented pigs tended to have a higher mean corpuscular haemoglobin concentration than CON pigs (28.88 vs 28.43 ± 0.186 g/dL; P=0.10).

Regarding platelet counts, a significant maternal effect was found on day 8 pw, with the offspring from CON sows having a lower platelet count than those from PRO sows (224.25 vs 332.28 ± 22.892 × 10³ cells/µL; P<0.01).

TABLE 9

Effect of supplementation with the spores from the deposited strain of the claimed invention to sow and piglet diets on haematological parameters of piglets at weaning and day 8, 27 and 57 post-weaning¹

| Maternal | | Control | Control | Probiotic | Probiotic | | P-value | | | | |
|---|---|---|---|---|---|---|---|---|---|---|---|
| Post-weaning (pw) | | Control | Probiotic | Control | Probiotic | | Maternal | pw | Maternal × pw | Day | Maternal × pw × Day |
| | Day (PW) | CON/CON 2 | CON/PRO 3 | PRO/CON 4 | PRO/PRO 5 | SEM | | | | | |
| White blood cells (×10³/µl) | 0^{‡} | 10.51 | 13.37 | 11.94 | 9.83 | 1.390 | 0.47 | 0.85 | 0.08 | | |
| | 8 | 12.76 | 11.92 | 13.63 | 10.22 | 1.555 | 0.74 | 0.17 | 0.40 | | |
| | 28 | 15.66 | 15.40 | 13.33 | 13.55 | 1.522 | 0.18 | 1.00 | 0.60 | | |
| | 57 | 10.34 | 14.05 | 13.20 | 15.23 | 1.363 | 0.13 | 0.04 | 0.08 | | |
| | Mean | 12.73 | 14.71 | 13.26 | 14.37 | 1.159 | 0.92 | 0.19 | 0.70 | 0.11 | 0.43 |
| Lymphocytes (×10³ cells/µL) | 0 | 5.12 | 6.56 | 6.11 | 5.76 | 1.121 | 0.94 | 0.63 | 0.43 | | |
| | 8 | 7.75 | 6.98 | 7.68 | 5.88 | 1.331 | 0.63 | 0.33 | 0.67 | | |
| | 28 | 10.44 | 10.75 | 8.67 | 9.76 | 1.667 | 0.41 | 0.68 | 0.81 | | |
| | 57 | 5.99 | 10.40 | 8.59 | 11.54 | 1.619 | 0.25 | 0.03 | 0.10 | | |
| | Mean | 8.21 | 10.57 | 8.63 | 10.65 | 1.162 | 0.83 | 0.06 | 0.89 | 0.51 | 0.63 |
| Lymphocytes (%)⁶ | 0 | 50.58 | 47.86 | 54.11 | 52.73 | 6.077 | 0.49 | 0.79 | 0.91 | | |
| | 8 | 57.96 | 53.29 | 57.04 | 55.48 | 5.473 | 0.90 | 0.57 | 0.78 | | |
| | 28 | 65.49 | 71.52 | 60.29 | 68.23 | 4.348 | 0.34 | 0.11 | 0.29 | | |
| | 57 | 59.30 | 66.80 | 59.37 | 69.26 | 4.180 | 0.76 | 0.04 | 0.22 | | |
| | Mean | 62.39 | 69.16 | 59.83 | 68.74 | 3.027 | 0.63 | 0.01 | 0.72 | 0.37 | 0.97 |
| Monocytes (× 10³ cells/µL) | 0 | 0.71 | 0.81 | 0.88 | 0.63 | 0.130 | 0.89 | 0.54 | 0.17 | | |
| | 8 | 0.45a | 0.77ab | 1.00^{b} | 0.58ab | 0.123 | 0.16 | 0.96 | <0.01 | | |
| | 28 | 0.83 | 0.67 | 0.76 | 0.69 | 0.135 | 0.85 | 0.42 | 0.86 | | |
| | 57 | 1.10 | 1.17 | 1.20 | 1.05 | 0.130 | 0.91 | 0.78 | 0.84 | | |
| | Mean | 0.96 | 0.92 | 0.98 | 0.87 | 0.094 | 0.83 | 0.44 | 0.72 | <0.001 | 0.41 |
| Monocytes (%)⁶ | 0 | 6.44 | 6.32 | 7.06 | 7.49 | 1.063 | 0.41 | 0.90 | 0.80 | | |
| | 8 | 3.78^{A} | 5.99^{AB} | 7.08^{B} | 6.24^{AB} | 0.955 | 0.05 | 0.32 | 0.09 | | |
| | 28 | 5.89 | 4.55 | 5.93 | 4.99 | 1.286 | 0.85 | 0.38 | 0.83 | | |
| | 57 | 11.34 | 8.59 | 9.97 | 7.30 | 1.236 | 0.29 | 0.03 | 0.13 | | |
| | Mean | 8.62 | 6.57 | 7.95 | 6.15 | 0.895 | 0.55 | 0.04 | 0.89 | <0.001 | 0.93 |
| Neutrophils (×10³ cells/µL) | 0 | 4.43^{AB} | 5.75^{A} | 4.66^{AB} | 3.21^{B} | 0.714 | 0.10 | 0.72 | 0.05 | | |
| | 8 | 4.36 | 3.99 | 3.85 | 3.62 | 0.519 | 0.40 | 0.58 | 0.92 | | |
| | 28 | 4.13 | 3.43 | 3.47 | 2.98 | 0.407 | 0.19 | 0.15 | 0.30 | | |
| | 57 | 2.85 | 3.07 | 3.21 | 2.78 | 0.392 | 0.93 | 0.79 | 0.86 | | |
| | Mean | 3.49 | 3.25 | 3.34 | 2.88 | 0.285 | 0.38 | 0.22 | 0.70 | 0.07 | 0.45 |
| Neutrophils (%)⁶ | 0 | 40.69 | 43.98 | 36.20 | 36.98 | 5.059 | 0.26 | 0.69 | 0.81 | | |
| | 8 | 36.81 | 38.02 | 34.10 | 36.84 | 4.676 | 0.68 | 0.67 | 0.86 | | |
| | 28 | 25.63 | 22.04 | 30.54 | 23.98 | 3.393 | 0.33 | 0.14 | 0.31 | | |
| | 57 | 25.15 | 21.09 | 26.27 | 20.49 | 3.270 | 0.94 | 0.14 | 0.51 | | |
| | Mean | 25.39 | 21.57 | 28.41 | 22.24 | 2.671 | 0.50 | 0.07 | 0.66 | 0.26 | 0.88 |
| Eosinophils (×10³ cells/µL) | 0 | 0.17 | 0.19 | 0.16 | 0.14 | 0.035 | 0.38 | 0.98 | 0.65 | | |
| | 8 | 0.16 | 0.13 | 0.17 | 0.09 | 0.024 | 0.42 | 0.03 | 0.29 | | |
| | 28 | 0.19 | 0.17 | 0.13 | 0.15 | 0.028 | 0.22 | 0.99 | 0.59 | | |
| | 57 | 0.19^{AB} | 0.14^{A} | 0.26^{B} | 0.15^{A} | 0.027 | 0.14 | <0.01 | 0.01 | | |
| | Mean | 0.19 | 0.15 | 0.20 | 0.15 | 0.019 | 0.888 | 0.05 | 0.71 | 0.19 | 0.19 |
| Eosinophils (%)⁶ | 0 | 1.63 | 1.31 | 1.47 | 1.63 | 0.291 | 0.78 | 0.77 | 0.41 | | |
| | 8 | 1.20 | 1.14 | 0.94 | 0.93 | 0.151 | 0.13 | 0.82 | 0.90 | | |
| | 28 | 1.15 | 1.09 | 0.94 | 0.89 | 0.206 | 0.34 | 0.78 | 0.81 | | |
| | 57 | 1.7^{2a}b | 0.89^{a} | 2.06^{b} | 1.02^{a} | 0.198 | 0.23 | <0.001 | 0.001 | | |
| | Mean | 1.43 | 0.99 | 1.50 | 0.95 | 0.145 | 0.903 | 0.001 | 0.72 | <0.01 | 0.71 |
| Basophils (× 10³ cells/µL) | 0 | 0.09 | 0.06 | 0.16 | 0.10 | 0.024 | 0.05 | 0.11 | 0.70 | | |
| | 8 | 0.03^{a} | 0.06^{b} | 0.08^{b} | 0.05^{ab} | 0.009 | 0.02 | 0.69 | 0.001 | | |
| | 28 | 0.23 | 0.15 | 0.26 | 0.21 | 0.039 | 0.27 | 0.13 | 0.27 | | |
| | 57 | 0.27 | 0.28 | 0.24 | 0.22 | 0.038 | 0.22 | 0.88 | 0.63 | | |
| | Mean | 0.25 | 0.22 | 0.25 | 0.22 | 0.027 | 0.95 | 0.23 | 0.98 | 0.13 | 0.57 |
| Basophils (%)⁶ | 0 | 0.66 | 0.51 | 1.15 | 1.16 | 0.230 | 0.01 | 0.64 | 0.61 | | |
| | 8 | 0.26^{a} | 0.54^{b} | 0.60^{b} | 0.51^{ab} | 0.085 | 0.03 | 0.11 | 0.02 | | |
| | 28 | 1.76 | 1.13 | 2.02 | 1.46 | 0.369 | 0.44 | 0.11 | 0.34 | | |
| | 57 | 2.56 | 2.10 | 1.92 | 1.56 | 0.355 | 0.11 | 0.24 | 0.26 | | |
| | Mean | 2.16 | 1.61 | 1.97 | 1.51 | 0.259 | 0.59 | 0.05 | 0.87 | 0.08 | 0.98 |
| Red blood cells (× 10⁶ cells/µL) | 0 | 7.94 | 7.71 | 7.02 | 6.93 | 0.450 | 0.07 | 0.72 | 0.88 | | |
| | 8 | 7.10 | 7.03 | 7.25 | 7.15 | 0.190 | 0.48 | 0.64 | 0.94 | | |
| | 28 | 7.03 | 7.20 | 7.09 | 7.16 | 0.173 | 0.96 | 0.48 | 0.90 | | |
| | 57 | 7.19 | 7.22 | 7.01 | 7.19 | 0.169 | 0.56 | 0.53 | 0.81 | | |
| | Mean | 7.11 | 7.21 | 7.05 | 7.18 | 0.151 | 0.77 | 0.45 | 0.93 | 0.68 | 0.44 |
| Haemoglobin (g/dL) | 0 | 15.36 | 14.79 | 13.38 | 13.90 | 0.840 | 0.10 | 0.98 | 0.52 | | |
| | 8 | 12.92 | 12.89 | 12.96 | 13.03 | 0.307 | 0.77 | 0.95 | 0.87 | | |
| | 28 | 12.10 | 12.45 | 12.26 | 12.72 | 0.280 | 0.45 | 0.15 | 0.47 | | |
| | 57 | 12.99 | 12.45 | 12.31 | 12.84 | 0.270 | 0.59 | 0.99 | 0.25 | | |
| | Mean | 12.55 | 12.45 | 12.29 | 12.78 | 0.195 | 0.86 | 0.31 | 0.13 | 0.18 | 0.22 |
| Haematocrit (L/L) | 0 | 0.51 | 0.49 | 0.44 | 0.46 | 0.024 | 0.05 | 0.98 | 0.55 | | |
| | 8 | 0.45 | 0.45 | 0.46 | 0.45 | 0.011 | 0.90 | 0.61 | 0.76 | | |
| | 28 | 0.40 | 0.42 | 0.41 | 0.42 | 0.010 | 0.48 | 0.15 | 0.48 | | |
| | 57 | 0.43 | 0.42 | 0.41 | 0.43 | 0.010 | 0.67 | 0.83 | 0.58 | | |
| | Mean | 0.42 | 0.42 | 0.41 | 0.43 | 0.009 | 0.87 | 0.33 | 0.53 | 0.20 | 0.15 |
| Mean corpuscular volume (fL) | 0 | 63.79 | 63.87 | 63.19 | 66.08 | 0.904 | 0.39 | 0.11 | 0.13 | | |
| | 8 | 64.02 | 64.34 | 63.04 | 62.72 | 1.151 | 0.27 | 0.99 | 0.78 | | |
| | 28 | 57.19 | 58.17 | 58.10 | 59.18 | 0.762 | 0.22 | 0.19 | 0.37 | | |
| | 57 | 59.81 | 57.71 | 58.64 | 59.39 | 0.747 | 0.73 | 0.37 | 0.23 | | |
| | Mean | 58.48 | 57.94 | 58.37 | 59.28 | 0.651 | 0.35 | 0.78 | 0.27 | 0.07 | 0.08 |
| Mean corpuscular haemoglobin (pg/cell) | 0 | 19.38 | 19.25 | 19.12 | 20.12 | 0.328 | 0.37 | 0.20 | 0.10 | | |
| | 8 | 18.22 | 18.42 | 17.86 | 18.26 | 0.325 | 0.43 | 0.36 | 0.75 | | |
| | 28 | 17.16 | 17.28 | 17.25 | 17.75 | 0.233 | 0.24 | 0.20 | 0.31 | | |
| | 57 | 18.08 | 17.25 | 17.50 | 17.83 | 0.230 | 0.98 | 0.28 | 0.08 | | |
| | Mean | 17.61 | 17.26 | 17.38 | 17.79 | 0.204 | 0.49 | 0.89 | 0.07 | <0.01 | 0.09 |
| Mean corpuscular haemoglobin concentration (g/dL) | 0 | 30.37 | 30.15 | 30.29 | 30.45 | 0.307 | 0.72 | 0.92 | 0.54 | | |
| | 8 | 28.50 | 28.65 | 28.37 | 29.12 | 0.264 | 0.53 | 0.10 | 0.27 | | |
| | 28 | 29.98 | 29.70 | 29.70 | 29.93 | 0.191 | 0.90 | 0.90 | 0.60 | | |
| | 57 | 30.25 | 29.87 | 29.87 | 30.04 | 0.184 | 0.57 | 0.58 | 0.42 | | |
| | Mean | 30.12 | 29.78 | 29.78 | 29.99 | 0.137 | 0.64 | 0.64 | 0.06 | 0.18 | 0.96 |
| Platelets (× 10³ cells/µL) | 0 | 318.10 | 351.50 | 426.70 | 406.80 | 50.627 | 0.11 | 0.90 | 0.60 | | |
| | 8 | 228.06 | 220.50 | 386.20 | 285.89 | 32.594 | <0.01 | 0.16 | 0.26 | | |
| | 28 | 362.07 | 371.50 | 349.61 | 345.03 | 34.682 | 0.58 | 0.95 | 0.94 | | |
| | 57 | 289.40 | 262.96 | 285.10 | 349.16 | 27.984 | 0.16 | 0.58 | 0.21 | | |
| | Mean | 323.70 | 312.55 | 315.71 | 347.09 | 25.040 | 0.61 | 0.70 | 0.41 | <0.01 | 0.15 |

¹ Least square means and pooled standard errors of the mean (SEM).
² CON/CON, non-probiotic supplemented sow/non-probiotic supplemented piglet;
³ CON/PRO, non-probiotic supplemented sow/probiotic-supplemented piglet;
⁴ PRO/CON, probiotic-supplemented sow/non-probiotic supplemented piglet;
⁵ PRO/PRO, probiotic-supplemented sow/probiotic-supplemented piglet.
⁶ Percentages are based on the differential count of white blood cells.
^{a-b} Values within a row that do not share a common superscript are significantly different (P≤0.05).
^{A-B} Values within a row that do not share a common superscript tended to differ (P≤0.10).
^{‡} Day 0 pw is the day of weaning.

Interestingly, some of the haematological parameters measured in sows indicate a possible inflammatory response after the first 2 weeks of probiotic treatment using the spores from the deposited strain of the claimed invention (day 114 of gestation), which persisted throughout the suckling period. Basophil counts in probiotic-supplemented sows were higher than those in control sows, although all values were within reference ranges, except the basophil percentage at weaning (the upper limit is 2.0% and the value in probiotic-supplemented sows was 2.32%). Probiotic-supplemented sows also had lower mean corpuscular volume and less mean corpuscular haemoglobin than control sows from farrowing to weaning but values were within the normal ranges, being indicative of subtle anaemia or possible inflammation. This possible immune modulation in the sow could have affected the pigs in utero (despite swine placenta being epitheliochorial), which may also help to explain the improved gut health early post-weaning and the subsequent growth benefits.

It is interesting to note that some of the haematological effects found in the sows were mirrored in the offspring. For example, piglets born to sows fed the probiotic comprising spores from the deposited strain of the claimed invention had higher basophil counts and percentages than the offspring from control sows on the day of weaning and at day 8 pw. This may have been caused by an in utero effect or it could be indicative of immune stimulation during the early stages of suckling due to early-life probiotic exposure. Nonetheless, this effect diminished after day 8 pw and was not observed thereafter. There was no effect of post-weaning treatment with the probiotic on the haematology of pigs; however, piglets that were never exposed to this isolated strain of the claimed invention had the lowest levels of basophils.

Other significant differences of note were the effects on WBC populations found due to probiotic administration post-weaning. These included elevated total WBC and lymphocyte counts and reduced monocyte and eosinophil levels, albeit all were within reference values. Interestingly, all were observed two months post-weaning (day 57 pw). However, these post-weaning treatment-related haematological effects did not translate into improved growth.

### Effects of Maternal And/or Post-Weaning Supplementation With Bacillus Altitudinis Spores on the Sow and Offspring Microbiota

All differentially abundant taxa are presented in Tables 10-13. However, only differentially abundant taxa with an abundance >1% in at least one treatment group are described in the text, with the exception of Bacillus.

### Effect of Maternal Treatment on Composition of the Sow Colostrum and Faecal Microbiota

The phylum Actinobacteria was increased in relative abundance in the colostrum of probiotic-supplemented sows compared with that of control sows (30.7% vs 21.9%; Table 8), likely resulting from an increased abundance of the genus Rothia (15.7 vs 6.9 %; Table 10). Rothia species are common oral, skin, and intestinal commensals of humans, pigs, and rodents and have previously been found in the milk of pigs, humans, goats and rats. The sequence identified as differentially abundant in the colostrum matches several Rothia spp., mainly those classified as environmental, but also one animal species and one species pathogenic for humans. However, Rothia infections are rare, and the human species are considered opportunistic pathogens, mostly affecting immunocompromised individuals.

TABLE 10

Effect of maternal treatment with spores from the deposited strain of the claimed invention on the % relative abundance of differentially abundant taxa in the colostrum (P<0.05)

| Taxa | Maternal Treatment | | SEM | P value |
|---|---|---|---|---|
| | Control | Probiotic | | |
| P- Actinobacteria | 21.94 | 30.73 | 1.940 | 0.025 |
| G- Rothia | 6.92 | 15.74 | 2.431 | 0.015 |
| G- Globicatella | 0.53 | 1.22 | 0.246 | 0.001 |
| G- Vagococcus | 0.50 | 0.01 | 0.198 | 0.001 |

P, phylum;
G, genus;
SEM, standard error of the mean

### Effect of Maternal/Post-Weaning Treatment on Offspring Faecal and Digesta Microbiota

This study was designed as a 2×2 factorial and the compositional results for all four treatment groups (Table 10) as well as the maternal treatment effect only (i.e. the effects in the offspring of probiotic-supplemented sows, irrespective of whether or not they were supplemented with probiotic themselves; Table 13) are shown. Overall, several differentially abundant genera and phyla were identified across the various sampling time points (Tables 11-13). The main findings are as follows:

### Weaning (Day 26)

At weaning, one differentially abundant phylum and 5 differentially abundant genera were identified in the offspring faeces (Table 11).

TABLE 11

Effect of maternal treatment with spores from the deposited strain of the claimed invention on the % relative abundance of differentially abundant taxa in the faeces of piglets at weaning (P<0.05)

| Taxa | Maternal Treatment | | SEM | P value |
|---|---|---|---|---|
| | Control | Probiotic | | |
| P- Synergistota | 2.608 | 0.763 | 0.4242 | 0.049 |
| G- Lachnospiraceae_NK4A136_group | 0.490 | 2.083 | 0.4483 | 0.013 |
| G- Rikenellaceae_dgA-11_gut_group | 2.049 | 0.215 | 0.3399 | <0.0001 |
| G- Prevotellaceae_UCG-003 | 0.712 | 0.153 | 0.1135 | <0.0001 |
| G- Muribaculaceae_CAG-873 | 0.621 | 0.126 | 0.1010 | 0.013 |
| G- Alloprevotella spp. | 2.496 | 1.240 | 0.3465 | 0.026 |

G, genus;
P, phylum;
SEM, standard error of the mean

Of interest, pigs weaned from the probiotic-supplemented sows had a higher abundance of Lachnospiraceae_NK4A136_group (2.1 vs 0.5%) compared with pigs weaned from the control sows. The Lachnospiraceae family are associated with fermentation of plant polysaccharides and the production of butyric acid, which is the preferential energy source of colonocytes. Butyric acid may enhance intestinal health by facilitating the absorption of water, sodium, and potassium, regulating gene expression by inhibiting the expression of histone deacetylases, enhancing the expression of host defence peptides, and antibacterial activity. Therefore, the increased abundance of Lachnospiraceae in the offspring from probiotic-supplemented sows may have benefits in terms of intestinal integrity and nutrient absorption. This may help to explain the positive modulation of gut histology parameters at day 8 pw in offspring from probiotic-supplemented sows, the improved FCE during the first 14 days post-weaning and the subsequent improvements in growth.

### Ileum Day 8 PW

The ileum was the intestinal site at which most effects were seen, with six differentially abundant phyla and 53 differentially abundant genera identified at day 8 pw (Table 12).

TABLE 12

Effect of maternal and post-weaning treatment on the % relative abundance of differentially abundant taxa in the digesta and faeces of pigs post-weaning (P<0.05)

| | Treatment | | | | SEM | P values | | | | | |
|---|---|---|---|---|---|---|---|---|---|---|---|
| Taxa | CON/CON | CON/PRO | PRO/CON | PRO/PRO | | CON/CON vs CON/PRO | CON/CON vs PRO/CON | CON/CON vs PRO/PRO | CON/PRO vs PRO/CON | CON/PRO vs PRO/PRO | PRO/CON vs PRO/PRO |
| Ileum D8 pw | | | | | | | | | | | |
| P- Actinobacteria | 2.917 | 0.878 | 0.709 | 0.611 | 0.2340 | 0.004 | 0.003 | | | | |
| P- Parescibacteria | 0.337 | 0.031 | 0.071 | 0.012 | 0.0600 | 0.014 | | 0.003 | | | |
| P- Bacteroidota | 4.401 | 8.032 | 13.780 | 2.021 | 1.2664 | | 0.006 | | | 0.004 | 0.001 |
| P- Firmicutes | 47.703 | 49.601 | 47.590 | 57.695 | 3.0284 | | | 0.017 | | | |
| P- Fusobacteria | 0.179 | 0.082 | 0.095 | 0.033 | 0.0280 | | | 0.004 | | | |
| P- Spirochaetota | 0.099 | 0.842 | 1.057 | 0.021 | 0.1744 | | 0.018 | | | | 0.0003 |
| G- Bacillus | 0.007 | 0.866 | 0.041 | 0.391 | 0.0915 | <0.001 | | 0.002 | | | |
| G- Chryseobacterium | 1.340 | 0.029 | 0.025 | 0.016 | 0.2544 | 0.002 | 0.013 | <0.001 | | | |
| G- Escherichialshigella | 3.033 | 20.517 | 6.806 | 12.801 | 2.4862 | 0.004 | 0.007 | <0.001 | | | |
| G- Prevotellaceae_UCG-001 | 0.026 | 0.206 | 0.250 | 0.042 | 0.0410 | 0.015 | 0.009 | | | | |
| G- Brevundimonas | 0.076 | 0.004 | 0.028 | 0.003 | 0.0103 | 0.021 | | | | | |
| G- Pelistega | 2.194 | 0.430 | 0.867 | 0.181 | 0.2786 | 0.044 | | | | | |
| G- Neisseria | 0.812 | 0.136 | 0.250 | 0.042 | 0.1016 | 0.015 | | 0.011 | | | |
| G- Rikenellaceae_RC9_gut_group | 0.153 | 0.925 | 1.181 | 0.131 | 0.1689 | 0.044 | 0.004 | | | | |
| G- Veillonella | 0.743 | 0.149 | 0.421 | 0.111 | 0.0747 | 0.021 | | | | | |
| G- Treponema | 0.089 | 0.769 | 0.969 | 0.021 | 0.1611 | 0.044 | 0.005 | | | | |
| G- Faecalibacterium | 0.059 | 0.143 | 0.485 | 0.138 | 0.0478 | | 0.004 | | | | |
| G- Christensenellaceae_R-7_group | 0.065 | 0.341 | 0.699 | 0.127 | 0.1125 | | 0.013 | | | | |
| G- Agathobacter | 0.113 | 0.263 | 0.470 | 0.174 | 0.0566 | | 0.013 | | | | |
| G-Allorhizobium-Neorhizobium-Pararhizobium-Rhizobium | 0.468 | 0.018 | 0.022 | 0.013 | 0.0530 | | 0.014 | <0.001 | | | |
| G- Roseburia | 0.020 | 0.077 | 0.223 | 0.090 | 0.0296 | | 0.018 | | | | |
| G- Fusicatenibacter | 0.015 | 0.016 | 0.080 | 0.006 | 0.0099 | | 0.018 | | | | |
| G- Corynebacterium | 0.292 | 0.067 | 0.042 | 0.039 | 0.0328 | | 0.022 | | | | |
| G- Blautia | 0.616 | 0.445 | 1.823 | 0.932 | 0.2811 | | 0.022 | 0.002 | | | |
| G- Megasphaera | 0.121 | 0.185 | 0.555 | 0.069 | 0.0640 | | 0.004 | | | | |
| G- Prevotella | 1.054 | 2.618 | 4.427 | 0.203 | 0.4863 | | 0.004 | | | | |
| G- Prevotellaceae_UCG-003 | 0.095 | 0.197 | 0.356 | 0.184 | 0.0505 | | 0.031 | | | | |
| G- Anaerovibrio | 0.108 | 0.197 | 0.577 | 0.057 | 0.0640 | | 0.006 | | | | |
| G- Butyricicoccaceae_UCG-008 | 0.170 | 0.318 | 0.855 | 0.343 | 0.1079 | | 0.004 | | | | |
| G- Oscillospiraceae_NK4A214_group | 0.025 | 0.188 | 0.249 | 0.030 | 0.0401 | | 0.009 | | | | |
| G- Oscillospiraceae_UCG-002 | 0.033 | 0.313 | 0.510 | 0.012 | 0.0796 | 0.044 | 0.004 | | | | 0.030 |
| G- Lachnospiraceae_AC2044_group | 0.014 | 0.044 | 0.095 | 0.042 | 0.0132 | | 0.026 | | | | |
| G- Lachnospiraceae_ND3007_group | 0.027 | 0.034 | 0.127 | 0.112 | 0.0189 | | 0.050 | | | | |
| G- Fournierella | 0.011 | 0.037 | 0.200 | 0.043 | 0.0242 | | 0.004 | | | | |
| G-Rikenellaceae_dgA-11_gut_group | 0.021 | 0.083 | 0.148 | 0.001 | 0.0171 | | 0.037 | | | | |
| G- Solobacterium | 0.037 | 0.045 | 0.125 | 0.077 | 0.0190 | | 0.023 | | | | |
| G- Turicibacter | 0.777 | 1.950 | 4.946 | 1.840 | 0.6849 | | 0.009 | | | | |
| G- Catenibacterium | 0.155 | 0.149 | 1.126 | 0.160 | 0.1600 | | 0.027 | | | | |
| G- Catenisphaera | 0.014 | 0.019 | 0.082 | 0.018 | 0.0091 | | 0.026 | | | | |
| G- Clostridium_sensu_stricto_6 | 0.015 | 0.050 | 0.114 | 0.214 | 0.0390 | | 0.007 | | | | |
| G- Rothia | 2.169 | 0.715 | 0.396 | 0.332 | 0.1876 | | 0.038 | 0.037 | | | |
| G- Phascolarctobacterium | 0.177 | 0.272 | 0.835 | 0.075 | 0.0762 | | 0.006 | | | | |
| G- Parabacteroides | 0.033 | 0.128 | 0.393 | 0.111 | 0.0418 | | 0.007 | | | | |
| G- Succinivibrio | 0.055 | 0.162 | 0.309 | 0.021 | 0.0500 | | 0.015 | | | | |
| G- Subdoligranulum | 0.090 | 0.180 | 0.445 | 0.340 | 0.0646 | | 0.007 | | | | |
| G- Collinsella | 0.035 | 0.054 | 0.142 | 0.130 | 0.0319 | | 0.007 | | | | |
| G- Camplyobacter | 0.127 | 0.132 | 0.604 | 0.540 | 0.1342 | | 0.025 | | | | |
| G- Alloprevotella | 0.432 | 0.549 | 1.522 | 0.292 | 0.1707 | | 0.050 | | | | 0.029 |
| G- Coprococcus | 0.068 | 0.123 | 0.447 | 0.193 | 0.0569 | | 0.007 | | | | |
| G- Ruminococcus | 0.024 | 0.195 | 0.270 | 0.085 | 0.0375 | | 0.010 | | | | |
| G- Sphaerochaeta | 0.010 | 0.072 | 0.084 | 0.000 | 0.0138 | | 0.022 | | | | |
| G- Holdemanella | 0.074 | 0.070 | 0.334 | 0.181 | 0.0477 | | 0.007 | | | | |
| G- Clostridium_sensu_stricto_1 | 5.096 | 6.649 | 4.275 | 12.229 | 1.6031 | | | 0.012 | | | |
| G- Prevotellaceae_NK3B31_group | 0.203 | 1.127 | 1.472 | 0.020 | 0.2126 | | 0.006 | | | 0.002 | 0.029 |
| G- Family_XIII_AD3011_group | 0.015 | 0.055 | 0.115 | 0.018 | 0.0174 | | 0.041 | | | | |
| G- Helcococcus | 0.060 | 0.040 | 0.033 | 0.002 | 0.0077 | | | 0.009 | | | |
| G- Enhydrobacter | 0.314 | 0.052 | 0.109 | 0.013 | 0.0487 | | | 0.037 | | | |
| G- Gemella | 0.206 | 0.052 | 0.089 | 0.024 | 0.0162 | | | 0.037 | | | |
| G- Terrisporobacter | 3.222 | 3.737 | 4.438 | 7.707 | 1.1116 | | | 0.037 | | | |
| Caecum D8 pw | | | | | | | | | | | |
| G- Christensenellaceae_R-7_ group | 0.065 | 0.227 | 0.412 | 0.197 | 0.0564 | | 0.034 | | | | |
| G- Treponema | 0.977 | 1.141 | 2.945 | 1.597 | 0.2675 | | 0.037 | | | | |
| G- Oscillospiraceae_NK4A214_group | 0.095 | 0.113 | 0.238 | 0.098 | 0.0241 | | 0.034 | | | | |
| Rectum D8 pw | | | | | | | | | | | |
| P- Campilobacterota | 0.962 | 2.847 | 1.045 | 0.987 | 0.2594 | 0.006 | | | 0.003 | 0.003 | |
| P- WPS-2 | 0.197 | 0.063 | 0.202 | 0.037 | 0.0420 | | | | 0.024 | | |
| Faeces D27 pw | | | | | | | | | | | |
| P- Firmicutes | 54.268 | 47.845 | 53.786 | 55.891 | 1.2625 | | | | 0.026 | 0.044 | |
| P- Actinobacteriota | 0.576 | 0.410 | 0.327 | 0.556 | 0.0452 | | | | | | 0.042 |
| P- Verrucomicrobiota | 0.362 | 0.497 | 0.479 | 0.112 | 0.0840 | | | | | | 0.006 |
| G- Lachnospiraceae_ND3007_group | 0.261 | 0.149 | 0.122 | 0.023 | 0.0220 | | | <0.001 | | 0.020 | |
| G- Prevotellaceae_UCG-001 | 0.365 | 0.646 | 1.126 | 0.937 | 0.2114 | | 0.005 | 0.002 | | | |
| G- Verrucomicrobiota_horsej-a03 | 0.017 | 0.043 | 0.002 | 0.001 | 0.0041 | | | | 0.028 | | |
| Faeces D56 pw | | | | | | | | | | | |
| G- Alloprevotella | 1.292 | 1.782 | 2.038 | 0.788 | 0.1451 | | | | | | 0.025 |
| Faeces D118 pw | | | | | | | | | | | |
| G- Lactobacillus spp. | 4.143 | 3.351 | 12.159 | 7.033 | 1.2104 | | 0.005 | | 0.007 | | |
| G- Muribaculaceae_CAG-873 | 0.236 | 0.535 | 0.093 | 0.124 | 0.0542 | | | | 0.007 | | |
| G- Succinivibrio | 1.026 | 1.154 | 0.257 | 0.674 | 0.2308 | | | | 0.038 | | |
| G- Catenisphaera | 0.057 | 0.142 | 0.029 | 0.041 | 0.0180 | | | | 0.007 | | |
| G- Lachnospiraceae_NK3A20_group | 0.062 | 0.097 | 0.027 | 0.084 | 0.016 | | | | 0.005 | | |
| G- Lachnospiraceae_NK4A136_group | 1.302 | 1.340 | 0.403 | 0.387 | 0.2402 | | | | 0.025 | | |
| G- Asteroleplasma | 0.166 | 0.300 | 0.124 | 0.088 | 0.0280 | | | | | 0.015 | |
| G- Roseburia | 0.160 | 0.117 | 1.043 | 0.770 | 0.1390 | | | | | | |

G, genus; P, phylum; SEM, standard error of the mean; D, day; pw, post-weaning.
Treatments: Maternal/Post-weaning; CON, control; PRO, probiotic.

The abundance of the Bacteroidota phylum was higher in the PRO/CON group compared with the CON/CON group (13.8% vs 4.4%). Bacteroidota abundance has previously been shown to increase post-weaning, likely resulting from the change in diet at this time. In the present study, it may be linked with the increase in members of the Prevotellaceae family (Prevotella, Alloprevotella and Prevotellaceae_NK3B31_group) in the PRO/CON group. Members of this family are involved in the fermentation of undigested carbohydrates and production of volatile fatty acids (VFA), thereby leading to increased energy harvest for the host. This increased abundance of members of the Prevotellaceae family may therefore help to explain the improved FCE during the first 14 days post-weaning in offspring from the probiotic-supplemented sows and the subsequent improvements in growth. Data on the role of Prevotella in the pig gut are conflicting but a recent review concluded that studies showing positive associations with feed efficiency and growth outnumber those demonstrating negative correlations. The genera Blautia and Turicibacter, which belong to the phylum Firmicutes were also increased in the PRO/CON group compared to the CON/CON group (1.8% vs 0.6% and 4.95% vs 0.78%, respectively). Blautia are also involved in carbohydrate fermentation and recently, Turicibacter was shown to be positively correlated with body weight in pigs.

The abundance of the phylum Firmicutes was increased in the PRO/PRO group compared with the CON/CON group (57.5% vs 47.7%), which may be considered a beneficial effect. This phylum is thought to contribute to VFA production and the regulation of systemic immune responses, contributing to energy salvage, the inhibition of opportunistic pathogens and protection against inflammation. Within this phylum, the genus Clostridium sensu stricto 1 was more abundant in the PRO/PRO compared with the CON/CON group (12.2% vs 5.1%). This genus has previously been found to be more abundant in more feed-efficient pigs, but in the faeces and caecum, although the opposite is also true. Terrisporobacter was also higher in abundance in the PRO/PRO group compared to the CON/CON group (7.7% vs 3.2%). This genus is commonly found at high abundance in the ileum of healthy pigs, although some species are considered emerging human pathogens.

At day 8 pw Bacillus (albeit detected at <1% abundance) was more abundant in the ileum of pigs supplemented with the probiotic post-weaning i.e., CON/PRO and PRO/PRO groups (the groups being fed probiotic at the time), than in pigs weaned from sows supplemented with the probiotic (PRO/CON) (0.87% and 0.39% vs 0.01%, respectively). This in in agreement with the Bacillus altitudinis probiotic count data at day 8 pw (see Table 13).

TABLE 13

Effect of spore supplementation of the isolated strain Bacillus altitudinis of the claimed invention to sow and piglet diets on ileal, 5 caecal and rectal digesta counts (log₁₀ CFU/g)¹ of piglets euthanized on day (D) 8 post-weaning

| Maternal | Control | Control | Probiotic | Probiotic | | | | | P-value | | |
|---|---|---|---|---|---|---|---|---|---|---|---|
| Post-weaning (pw) | Control | Probiotic | Control | Probiotic | | Maternal | pw | Maternal x pw | | Day | Maternal × pw × Day |
| | CON/CON² | CON/PR0³ | PRO/CON⁴ | PRO/PRO⁵ | SEM | | | | | | |
| N | 10 | 10 | 10 | 10 | | | | | | | |
| Ileum (D8 pw) | 3.00⁶ | 5.13 | 3.00 | 5.13 | 0.153 | 0.99 | <0.001 | 0.99 | | | |
| Caecum (D8 pw) | 3.00 | 5.48 | 3.00 | 5.37 | 0.114 | 0.62 | <0.001 | 0.62 | | | |
| Rectum (D8 pw) | 3.00 | 5.97 | 3.00 | 6.07 | 0.065 | 0.44 | <0.001 | 0.44 | | | |

¹ Least square means and pooled standard errors of the mean (SEM).
² CON/CON, non-probiotic supplemented sow/non-probiotic supplemented piglet;
³ CON/PRO, non-probiotic supplemented sow/probiotic-supplemented piglet;
⁴ PRO/CON, probiotic-supplemented sow/non-probiotic supplemented piglet;
⁵ PRO/PRO, probiotic-supplemented sow/probiotic-supplemented piglet.
⁶ The limit of detection of the assay for B. altitudinis WIT588 was 1000 CFU/g faeces. Values below the limit of detection were recorded as 3.00 log₁₀ CFU/g faeces.

The results in Table 13 show that the probiotic of the claimed invention was detected in the ileum, caecum and rectum of the CON/PRO and PRO/PRO groups, i.e. the pigs fed the probiotic during the post-weaning period.

The abundance of the genus Escherichia/Shigella was higher in all three probiotic-supplemented groups (20.5%, 6.8% and 12.8%, respectively vs 3.0% in the CON/CON group). However, the opposite was true when the maternal effect was examined, with pigs weaned from probiotic-supplemented sows having lower abundance of Escherichia/Shigella than those weaned from control sows (9.96% vs 11.32%; Table 11). However, it is important to note that the latter is due to the very high abundance of this genus in the CON/PRO group. Furthermore, there was large animal-to-animal variation within treatment groups. The genus Escherichia/Shigella has been shown to be more abundant in pigs with diarrhoea, but in rectal samples and during suckling, rather than post-weaning. On the other hand, Escherichia/Shigella is generally abundant in the ileum of healthy pigs, i.e. up to 23% abundance in some studies. Shigella spp. are not known to cause disease in pigs. Furthermore, the predominant E. coli serotype causing post-weaning diarrhoea in pigs is 0149 and this was not identified in the BLAST search of the sequence identified in this study. While one of the sequence matches, E. coli 0157:H7, is a food-borne pathogen, which causes disease in humans and may be carried by pigs, it is not associated with porcine disease. Furthermore, diarrhoea was not observed in any of the treatment groups in the present study and villous height was improved in the duodenum and ileum of pigs weaned from the probiotic-supplemented sows, suggesting that the E. coli detected are commensal rather than pathogenic to pigs.

### Caecum Day 8 PW

Three differentially abundant genera were identified in the caecum. Of interest, the abundance of Treponema was increased in the PRO/CON compared with the CON/CON group (2.95% vs 0.98%). This genus is involved in the breakdown of cellulose and lignin and is associated with improved feed efficiency in pigs. Its increased abundance may therefore also help to explain the improved FCE during the first 14 days post-weaning in offspring from the probiotic-supplemented sows and the subsequent improvements in growth. There was also a maternal treatment effect on the phylum Firmicutes, whereby pigs weaned from PRO sows had a lower abundance of Firmicutes; however, this reduction was very small (46.9 vs 45.5%; Table 14).

TABLE 14

Effect of maternal treatment with spores from the deposited strain of the claimed invention on the % relative abundance of differentially abundant taxa in the digesta and faeces of piglets post-weaning (P<0.05)

| Taxa | Maternal Treatment | | SEM | P value |
|---|---|---|---|---|
| | Control | Probiotic | | |
| Ileum D8 pw | | | | |
| G- Acinetobacter | 0.308 | 0.132 | 0.0699 | 0.016 |
| G- Aeromonas | 1.935 | 2.206 | 0.7366 | 0.027 |
| G- Arcanobacterium | 0.017 | 0.005 | 0.0032 | 0.031 |
| G- Brevundimonas | 0.042 | 0.015 | 0.0103 | 0.003 |
| G- Muribaculaceae_CAG-873 | 0.012 | 0.006 | 0.0029 | 0.025 |
| G- Fibrobacter | 0.062 | 0.057 | 0.0259 | <0.001 |
| G-Allorhizobium-Neorhizobium-Pararhizobium-Rhizobium | 0.254 | 0.017 | 0.0530 | <0.001 |
| G- Chryseobacterium | 0.719 | 0.020 | 0.2544 | 0.002 |
| G- Conchiformibus | 0.197 | 0.062 | 0.0490 | 0.025 |
| G- Corynebacterium | 0.185 | 0.040 | 0.0328 | 0.025 |
| G- Enhydrobacter | 0.189 | 0.052 | 0.0487 | 0.046 |
| G-dgA-11_gut_group | 0.051 | 0.063 | 0.0171 | 0.049 |
| G- Neisseria | 0.492 | 0.141 | 0.1016 | 0.003 |
| G- Escherichia/Shigella | 11.315 | 9.961 | 2.4862 | 0.017 |
| G- Pelistega | 1.358 | 0.506 | 0.2786 | 0.022 |
| G- Prevotellaceae_NK3B1_group | 0.641 | 0.708 | 0.2126 | 0.049 |
| G- Pseudomonas | 0.081 | 0.027 | 0.0132 | <0.001 |
| G- Gemella | 0.124 | 0.055 | 0.0162 | 0.001 |
| G- Globicatella | 0.079 | 0.036 | 0.0117 | 0.027 |
| G- Helcococcus | 0.051 | 0.016 | 0.0077 | 0.003 |
| G- Veillonella | 0.462 | 0.258 | 0.0747 | 0.016 |
| G- Rothia | 1.442 | 0.362 | 0.1876 | 0.006 |
| G- Bacillus | 0.414 | 0.225 | 0.0915 | 0.024 |
| G- Patescibacteria | 0.192 | 0.040 | 0.0600 | 0.007 |
| G- Verrucomicomicrobiota | 0.093 | 0.342 | 0.0868 | 0.022 |

Rectum D8 pw

| G- Butyricimonas | 0.011 | 0.055 | 0.0142 | <0.001 |
|---|---|---|---|---|
| G- Acidaminococcus | 0.360 | 0.057 | 0.0770 | 0.015 |
| G- Phocea | 0.002 | 0.048 | 0.006 | 0.015 |
| Caecum D8 pw | | | | |
| P- Firmicutes | 46.897 | 45.456 | 1.1976 | 0.009 |
| G- Anaerovorax | 0.004 | 0.037 | 0.0127 | 0.009 |
| Faeces D27 pw | | | | |
| G- Lachnospiraceae_ND3007_group | \| 0.202 \| | 0.075 \| | 0.0220 | \| <0.001 |

G, genus; P, phylum; SEM, standard error of the mean; D, day; pw, post-weaning.

### Faeces Day 27 PW

Three genera and three phyla were differentially abundant in the faeces on day 27 pw. Of note, the abundance of Prevotellaceae_UCG-001 was higher in the PRO/CON group compared with the CON/CON group (1.1% vs 0.4%). This reflects findings for other members of the Prevotellaceae family in the ileum at day 8 pw. As Prevotellaceae may be involved in increased energy harvest for the host, this increased Prevotellaceae_UCG-001 abundance may help to explain the subsequent improvements in growth in offspring from the probiotic-supplemented sows.

### Faeces Day 118 PW

There were eight differentially abundant genera in the faeces on day 118 pw. Most notably, Lactobacillus was more abundant in the PRO/CON group compared with the CON/CON and CON/PRO groups (12.2% vs 4.1% and 3.4% respectively). Lactobacillus spp. are considered beneficial and may enhance gut health through the competitive exclusion of pathogens, antioxidant activity and immunomodulation. Furthermore, Lactobacillus is consistently enriched in the large intestine of more feed-efficient pigs across studies. The genus Roseburia was also increased in abundance in the PRO/CON group compared with the CON/CON group (1.0% vs 0.2%). Roseburia spp. are associated with the fermentation of a range of substrates and butyrate production, with possible associated anti-inflammatory effects. They have also been associated with improved feed efficiency. Increased abundance of both genera in the offspring of the probiotic-supplemented sows at the end of the finisher period may help to help the improved growth in these animals at this stage.

To summarise, in the offspring, most effects on the microbiota were observed in the ileum on day 8 pw. While most differences were in genera which are lowly abundant, probiotic supplementation did affect some high abundance taxa. Most notably, it was associated with increased Escherichia/Shigella, but these were most likely commensal. Furthermore, there was an increase in the phylum Bacteroidota and some genera involved in polysaccharide fermentation in the PRO/CON group, and an increase in Firmicutes in the PRO/PRO group in the ileum on day 8 pw. These effects can be considered beneficial and may help to explain the improved feed efficiency in the offspring of probiotic-supplemented sows during this early post-weaning period. Another finding of note was the increase in the beneficial genera Lactobacillus and Roseburia in the PRO/CON group at day 118 pw; these increases may have contributed to the improved growth performance observed in pigs weaned from the probiotic-supplemented sows during the finishing period.

### Enzyme Production of Isolated Bacillus Altitudinis Strain as Deposited with the National Collection of Industrial and Marine Bacteria Under the Accession No. NCIMB 43558 on 27/01/2020

In vitro agar plate assays were used to test the isolated Bacillus altitudinis strain of the claimed invention for production of a range of extracellular enzymes, for example, proteases, cellulases, xylanases, phytases, β-glucanase and amylases. The isolated strain was shown to produce all of these enzymes, except for xylanase. Production of these enzymes could aid in the digestion of feed, thereby releasing additional energy for growth of the monogastric animal or monogastric mammal.

TABLE 15

Averaged zones of clearance produced by probiotic Bacillus strains after spotting on media containing substrate indicating exogenous enzyme production.

| Probiotic Test Strain | Mean Zone of Clearance¹ | | | | | |
|---|---|---|---|---|---|---|
| | Protease | Cellulase | Xylanase | a-glucanase | Amylase | Phytase³ |
| B. subtilis C-3102 from Calsporin | +++ (1.51)² | +++ (1.02) | ++ (2.73) | ++++ (1.39) | ++++ (1.18) | ++ |
| Bacillus altitudinis* | ++ (1.21) | + (0.71) | | ++++ (1.34) | ++ (0.24) | ++ |
| B. licheniformis DSM 5749 from Bioplus 2B | +++ (1.77) | ++ (1.77) | ++ (1.53) | ++++ (1.01) | ++++ (1.18) | ++ |
| B. subtilis DSM 5750 from Bioplus 2B | ++ (1.84) | + (0.49) | | ++++ (1.26) | ++ (1.89) | ++ |

*Strain of the claimed invention, as deposited with the National Collection of Industrial and Marine Bacteria under the Accession No. NCIMB 43558 on 27/01/2020.
¹ Diameter of zones of clearance in mm excluding culture spot (values averaged across triplicate assays conducted in triplicate): 0 mm (-); 0.1-3 mm (+); 3.1-6 mm (++); 6.1-9 mm (+++); 9.1-12 mm (++++).
² Standard deviation of triplicate assays conducted in triplicate.
³ Phytase data are based on a single triplicate assay conducted on one occasion only.

### Summary

In conclusion, this study indicated that the deposited strain of the claimed invention survived well in the GI tract of pigs, even in piglets not directly supplemented with the probiotic themselves, but whose mothers were administered probiotic. The faecal probiotic count in the piglets indicates that the probiotic of the strain of the claimed invention had transferred successfully from the sow to offspring, most likely via the faecal-oral route. While not wanting to be bound by theory, the Applicants hypothesise that probiotic spores of the strain of the claimed invention that are excreted in the sow’s faeces germinate in the sow faeces and/or in the farrowing house environment and, due to the relatively high gastric pH in suckling piglets, survive gastric transit as vegetative cells in the piglets leading to early colonization of the gut. This early colonization may help to explain the beneficial effects observed in these animals and not in piglets to which the probiotic spores of the strain of the claimed invention are administered post-weaning, as it appears that the spores of the strain of the claimed invention may not germinate in the gut. These beneficial effects include an improvement in FCR during the period from day 0 to 14 pw in piglets weaned from the sows supplemented with probiotic (see Table 4), indicating improved gut health. Histological data backs this up, indicating increased absorptive capacity in the small intestine at day 8 pw (see Table 7 and FIG. 2). A residual effect of probiotic supplementation comprising the strain of the claimed invention to sows was also observed in the offspring at day 105 pw and day 127 pw where heavier live weights were observed (see Table 4). Moreover, carcass weight and kill out percentages were higher in pigs from sows supplemented with probiotic comprising the strain of the claimed invention (see Table 5). Thus, administration of the probiotic comprising the strain of the claimed invention to sows during late gestation and lactation, rather than direct administration to the piglets themselves, is beneficial to offspring.

The data presented here suggests that the strain of the claimed invention leads to, amongst other beneficial effects, an improvement of colostrum quality, maternal immunomodulation, which was mirrored to a certain extent in the offspring, increased small intestinal absorptive capacity in offspring early post-weaning and modulation of colostrum microbiome and offspring intestinal (especially ileal) microbiota. Considering all these, supplementation of probiotic spores of the strain of the claimed invention to the sows is a cost-effective means of probiotic delivery to piglets to ensure early life colonization, improve gut health at the critical time of weaning and ultimately to increase pig carcass weight at their target slaughter age.

In the specification the terms “comprise, comprises, comprised and comprising” or any variation thereof and the terms “include, includes, included and including” or any variation thereof are considered to be totally interchangeable and they should all be afforded the widest possible interpretation and vice versa.

The invention is not limited to the embodiments hereinbefore described but may be varied in both construction and detail.

1. An isolated Bacillus altitudinis strain as deposited with the National Collection of Industrial and Marine Bacteria under the Accession No. NCIMB 43558 on 27/01/2020.

2. A composition comprising the isolated Bacillus altitudinis strain of claim 1.

3. The composition of claim 2, wherein the composition is a food product, a beverage product, a nutritional supplement, a drinking water additive, or an animal feed additive.

4. The composition of any one of claims 2 or 3, wherein the composition further comprises a probiotic material.

5. The composition of any one of claims 2 to 4, further comprising a prebiotic material.

6. The composition of any one of the preceding Claims, further comprising vitamins, minerals, chemical preservatives, antibiotics, fermentation products, proteins, carbohydrates, fats, enzymes, immune modulators, milk replacers, amino acids, coccidiostats, acid-based products and/or medicines, such as antibiotics, and other essential ingredients.

7. The composition of any one of the preceding claims, further comprising a suitable feed additive carrier.

8. The composition of claim 7, wherein the feed additive carrier is selected from the group comprising anti-caking agents, anti-oxidation agents, bulking agents, and/or protectants.

9. The composition of any one of claims 2 to 8, wherein the isolated Bacillus altitudinis strain is viable or non-viable.

10. The composition of any one of claims 2 to 9, wherein the composition comprises at least 10⁶ cfu or spores of the isolated Bacillus altitudinis strain per ml of composition.

11. A composition according to any one of claims 1 to 10 for use in a method of increasing weight gain in offspring of a monogastric animal fed or exposed to the composition.

12. A composition according to any one of claims 1 to 10 for use in a method of improving the gastrointestinal health of a subject fed or exposed to the composition.

13. A pharmaceutical composition comprising the isolated Bacillus altitudinis strain of claim 1, and a suitable pharmaceutical carrier.

14. The pharmaceutical composition of claim 13, wherein the composition is provided in a unit dose form suitable for oral administration, topical administration, nasopharyngeal administration, or environmental administration.

15. A pharmaceutical composition according to any one of claims 11 or 12 for use in a method of increasing weight gain in offspring of a monogastric animal fed or exposed to the composition.

16. A pharmaceutical composition according to any one of claims 11 or 12 for use in a method of improving the gastrointestinal health of a subject fed or exposed to the composition.

17. A composition comprising spores from the isolated Bacillus altitudinis strain of claim 1 for use in improving the nutritional quality of colostrum, positively modulating the histology parameters of the intestine, and positively modulating the intestinal and/or colostrum microbiome.

18. A composition comprising spores from the isolated Bacillus altitudinis strain of claim 1 for use in positively modulating the immune system of a subject and/or their offspring subjected to the composition.

19. A cell extract or supernatant of an isolated Bacillus altitudinis strain as deposited with the National Collection of Industrial and Marine Bacteria under the Accession No. NCIMB 43558 on 27/01/2020.

20. A composition comprising the cell extract or supernatant of the isolated Bacillus altitudinis strain of claim 19.

21. The composition of claim 20, wherein the composition is a food product, a beverage product, a nutritional supplement, a drinking water or liquid refreshment additive, or an animal feed additive.

22. The composition of any one of claims 20 or 21, wherein the composition comprises a probiotic material and optionally a prebiotic material.

23. The composition of any one of claims 20 to 22, further comprising vitamins, minerals, chemical preservatives, fermentation products, proteins, carbohydrates, fats, additional probiotics, prebiotics, enzymes, immune modulators, milk replacers, amino acids, coccidiostats, acid-based products and/or medicines, such as antibiotics.

24. The composition of any one of claims 20 to 23, further comprising a suitable feed additive carrier.

25. The composition of claim 24, wherein the feed additive carrier is selected from the group comprising anti-caking agents, anti-oxidation agents, bulking agents, and/or protectants.

26. The composition of any one of claims 20 to 25, wherein the isolated Bacillus altitudinis strain is viable or non-viable.

27. The composition of any one of claims 20 to 26, wherein the composition comprises at least 10⁶ cfu or spores of the isolated Bacillus altitudinis strain per ml of composition.

28. A feed additive comprising the isolated Bacillus altitudinis strain of claim 1, the cell extract or supernatant of the isolated Bacillus altitudinis strain of claim 19, the composition of claim 2, the composition of claim 20, or a combination thereof, for use in a method of increasing weight gain in offspring of a monogastric animal fed or exposed to the same.

29. A pharmaceutical composition comprising the cell extract or supernatant of the isolated Bacillus altitudinis strain of claim 20, and a suitable pharmaceutical carrier.

30. The pharmaceutical composition of claim 29, wherein the composition is provided in a unit dose form suitable for oral administration, nasopharyngeal administration, topical administration, or environmental administration.

31. A pharmaceutical composition according to any one of claims 29 to 30 for use in a method of increasing weight gain in offspring of a monogastric animal fed or exposed to the composition.

32. A method of increasing weight gain in an offspring of a monogastric animal or monogastric mammal, the method comprising providing the monogastric animal or monogastric mammal with the composition of any one of claims 2 to 10 or claims 20 to 27 during late gestation and following birth.

33. The method of claim 32, wherein the monogastric animal or monogastric mammal is fed the composition, or the composition is applied to a portion of the body of the monogastric animal or monogastric mammal, or the composition is dispersed into the environment of the monogastric animal or monogastric mammal for about the final 14 days of gestation and for all of the lactating period or about the first 28 days following birth, or for the entire gestation period and for about the first 28 days following birth.

34. A method of increasing weight gain in an offspring according to claim 32 or claim 33, wherein the monogastric animal or the monogastric mammal is a lactating mammal and the composition is provided during late gestation and during the lactating period following birth.

35. The method of claim 33 or claim 34, wherein the portion of the body of the monogastric mammal is selected from an udder, a teat, a flank, or any combination thereof.

36. A method of increasing weight gain in an offspring of a bird, the method comprising injecting the composition of any one of claims 2 to 10 or claims 20 to 27 in ovo prior to hatching.

37. A method of positively modulating the histology parameters of the intestine and positively modulating the intestinal microbiome of a bird, the composition comprising injecting the composition of any one of claims 2 to 10 or claims 20 to 27 in ovo prior to hatching.

38. A method of positively modulating the immune system of a bird and/or their offspring, the method comprising exposing the bird to the composition of any one of claims 2 to 10 or claims 20 to 27.

39. A method of increasing commensal bacteria in the gut of a monogastric animal or mammal and/or in the gut of offspring of a monogastric animal or mammal, the method comprising feeding the monogastric animal or mammal the composition of any one of claims 2 to 10 or claims 20 to 27, or applying the composition of any one of claims 2 to 10 or claims 20 to 27 to a portion of the body of the monogastric animal or mammal, or applying the composition of any one of claims 2 to 10 or claims 20 to 27 to the environment of the monogastric animal or mammal.

40. A method of increasing commensal bacteria in the colostrum of a monogastric animal or mammal, the method comprising feeding the monogastric animal or mammal the composition of any one of claims 2 to 10 or claims 20 to 27, or applying the composition of any one of claims 2 to 10 or claims 20 to 27 to the environment of the monogastric animal or mammal.

41. A composition for use in increasing adherence of commensal bacteria in the gut, the composition comprising the isolated Bacillus altitudinis strain, or spores thereof, or a combination thereof, as claimed in claim 1.
